(19) United States
(12) Patent Application Publication
(10) Pub. No.: US 2015/0232828 A1
(43) Pub. Date: Aug. 20, 2015

(54) METHOD FOR THE MANUFACTURING OF RECOMBINANT PROTEINS HARBOURING AN N-TERMINAL LYSINE

(71) Applicant: MERZ PHARMA GmbH & CO. KGaA, FRANKFURT am MAIN (DE)

(72) Inventors: Jurgen Frevert, Berlin (DE); Michael Schmidt, Potsdam (DE); Fred Hofmann, Potsdam (DE); Gerhard Groer, Potsdam (DE)

(21) Appl. No.: 14/422,509

(22) PCT Filed: Aug. 20, 2013
(86) PCT No.: PCT/EP2013/002506

§ 371 Date: Feb. 19, 2015

Related U.S. Application Data
(60) Provisional application No. 61/684,948, filed on Aug. 20, 2012.

(30) Foreign Application Priority Data
Aug. 20, 2012 (EP) 12005953.0

(51) Int. Cl. C12N 9/52

(52) U.S. Cl. CPC C12N 9/52; A61K 38/00

(57) ABSTRACT

This invention relates to a novel method for manufacturing and obtaining recombinant proteins, such as clostridial neurotoxins, harbouring an N-terminal lysine from precursor proteins. The method comprises the step of expressing a nucleic acid sequence encoding a precursor protein comprising an N-terminal motif, which can be recognised by an endoprotease specific for a lysine in P′1 position, and the step of cleaving the precursor protein with the endoprotease. The invention further relates to novel precursor proteins used in such methods, nucleic acid sequences encoding such precursor proteins and novel recombinant proteins, such as clostridial neurotoxins, harbouring an N-terminal lysine.

### FIELD OF THE INVENTION

This invention relates to a novel method for manufacturing and obtaining recombinant proteins, such as clostridial neurotoxins, harbouring an N-terminal lysine from precursor proteins. The method comprises the step of expressing a nucleic acid sequence encoding a precursor protein comprising an N-terminal motif, which can be recognised by an endoprotease specific for a lysine in P′1 position, and the step of cleaving the precursor protein with said endoprotease. The invention further relates to novel precursor proteins used in such methods, nucleic acid sequences encoding such precursor proteins and novel recombinant proteins, such as clostridial neurotoxins, harbouring an N-terminal lysine.

### BACKGROUND OF THE INVENTION

The amino acid methionine is encoded by a single codon, namely AUG, in the standard genetic code. The codon AUG is also the common start codon that signals the initiation of protein translation. Therefore the protein synthesis is commonly started with methionine, which is incorporated into the N-terminal position of all proteins in eukaryotes and archea. In bacteria, the derivative N-formylmethionine (fMet), in which a formyl group is added to the amino group of methionine, is used as the initial amino acid. fMet is coded by the same codon as methionine, AUG. When the codon is used for translation initiation, fMet is used, forming the first amino acid of the nascent polypeptide chain. When the same codon appears further downstream in the mRNA, normal methionine is incorporated.

In about two thirds of proteins the initial methionine or N-formylmethionine, respectively, is excised post-translationally.

The N-terminal methionine excision is catalyzed by methionyl-aminopeptidase (MAP), depending on the nature of the second amino acid residue in the polypeptide chain. In bacteria the N-formyl group has to be removed first by the peptide deformylase (PDF). N-terminal methionine excision (NME) is mainly responsible for the diversity of N-terminal amino acids in proteins. As a result of NME, Gly, Ala, Pro, Cys, Ser, Thr or Val residues may be found at the N-terminus of proteins, in addition to Met. If the second amino acid is lysine, NME does not occur.

NME is a conserved pathway essential in bacteria and lower eukaryotes. Dedicated NME components have been identified in all organisms. By determining the N-terminal amino acid in polypeptides, NME plays an important role in controlling protein turnover.

The N-terminal amino acid of a protein is an important factor governing its half-life, a rule that is referred to as N-end rule. The N-end rule is related to ubiquitination and proteasomal degradation and is applicable to both eukaryotic and prokaryotic organisms, but to a different extent. Although the proteolytic machineries differ in prokaryotes and eukaryotes, the principles of substrate recognition are conserved. In eukaryotes substrate recognition is mediated by N-recognins, a class of E3 ligases that label substrates via covalent linkage to ubiquitin, allowing the subsequent proteasomal degradation. In bacteria, the adaptor protein ClpS, which exhibits homology to the substrate-binding site of N-recognin, binds to the destabilizing N-termini of substrates and directly transfers them to the ClpAP protease.

The impact of the N-terminal amino acid on the protein turnover depends on the organism and can be modulated by N-terminal amino acid modification. Furthermore, additional degradation signals, known as degrons, can be found in polypeptide sequences, obscuring estimations of protein half-life based on the N-end rule. Valine, methionine, glycine, proline, threonine, and alanine are generally considered to be stabilising, whereas arginine, lysine, phenylalanine, aspartate, tyrosine, tryptophan, glutamine, and glutamate are considered to be destabilising when present at the N-terminal position of a protein.

Cellular proteins differ greatly regarding their half-life. Proteolytic degradation eliminates abnormal proteins, maintains the pool of free amino acids in cells affected by stresses such as starvation, and allows for generation of biologically active protein fragments that function as hormones, antigens or other effectors. Metabolic instability is a property of many regulatory proteins, whose concentration must vary with time and the state of the cell. A short protein half-life allows for the generation of spatial gradients and rapid adjustments of protein levels.

The majority of recombinant proteins that are obtained by expression in bacteria such as E. coli harbour an N-terminal formylmethionine. The removal of the N-terminal translation initiator fMet is often crucial for the function of the recombinant protein and allows for modulation of protein stability. Furthermore, in the human body fMet triggers an immune response.

As the methionyl-aminopeptidase (MAP) does not enzymatically excise the N-terminal fMet if the second amino acid residue is lysine, recombinant proteins with an N-terminal lysine are not obtainable so far. However, as lysine is a destabilising amino acid when present at the N-terminus, the generation of recombinant proteins with an N-terminal lysine might be advantageous, especially for pharmaceutical recombinant proteins that are potentially harmful and whose biological activity in the human body has therefore to be tightly regulated.

In recent years, botulinum neurotoxins have been used as therapeutic agents in the treatment of dystonias and spasms. Since clostridial toxins are highly toxic, there is a strong demand to produce the toxins with the highest possible purity and reproducibility and to obtain clostridial neurotoxins with tightly regulated biological activity upon administration to humans.

Clostridium is a genus of obligate anaerobe gram-positive bacteria, consisting of around 100 species that include important pathogens, such as Clostridium botulinum and Clostridium tetani. Both species produce neurotoxins, botulinum toxin and tetanus toxin, respectively. These neurotoxins are potent inhibitors of calcium-dependent neurotransmitter secretion of neuronal cells and are among the strongest toxins known to man. The lethal dose in humans lies between 0.1 ng and 1 ng per kilogram of body weight.

Oral ingestion of botulinum toxin via contaminated food or generation of botulinum toxin in wounds can cause botulism, which is characterised by paralysis of various muscles. Paralysis of the breathing muscles can cause death of the affected individual.

Both botulinum neurotoxin (BoNT) and tetanus neurotoxin (TxNT) inhibit neurotransmitter release from the axon of the affected neuron into the synapse. While the botulinum toxin acts at the neuromuscular junction and other cholinergic synapses in the peripheral nervous system, inhibiting the release of the neurotransmitter acetylcholine, the tetanus toxin acts mainly in the central nervous system. There it prevents the release of the inhibitory neurotransmitters, which leads to muscle overactivity resulting in generalized contractions of the agonist and antagonist musculature, termed a tetanic spasm.

While the tetanus neurotoxin exists in one immunologically distinct type, the botulinum neurotoxins are known to occur in seven different immunogenic types, termed BoNT/A through BoNT/G. Most Clostridium botulinum strains produce one type of neurotoxin but strains producing multiple toxins have also been described.

Botulinum and tetanus neurotoxins have highly homologous amino acid sequences and show a similar domain structure. Their biologically active form comprises two peptide chains, a light chain of about 50 kDa and a heavy chain of about 100 kDa, linked by a disulfide bond. A linker or loop region, whose length varies among different clostridial toxins, is located between the two cysteine residues forming the disulfide bond. This loop region is proteolytically cleaved by an unknown clostridial protease to obtain the biologically active toxin.

The molecular mechanism of intoxication by TxNT and BoNT appears to be similar as well: entry into the target neuron is mediated by binding of the C-terminal part of the heavy chain to a specific cell surface receptor; the toxin is then taken up by receptor-mediated endocytosis. The low pH in the so formed endosome then triggers a conformational change in the clostridial toxin which allows it to embed itself in the endosomal membrane and to translocate through the endosomal membrane into the cytoplasm, where the disulfide bond joining the heavy and the light chain is reduced. The light chain can then selectively cleave so called SNARE-proteins, which are essential for different steps of neurotransmitter release into the synaptic cleft, e.g. recognition, docking and fusion of neurotransmitter-containing vesicles with the plasma membrane. TxNT, BoNT/B, BoNT/D, BoNT/F, and BoNT/G cause proteolytic cleavage of synaptobrevin or VAMP (vesicle-associated membrane protein), BoNT/A and BoNT/E cleave the plasma membrane-associated protein SNAP-25, and BoNT/C cleaves the integral plasma membrane protein syntaxin and SNAP-25.

In recent years, botulinum neurotoxins have been used as therapeutic agents in the treatment of dystonias and spasms. Preparations comprising botulinum toxin complexes are commercially available, e.g. from Ipsen Ltd (Dysport®) or Allergan Inc. (Botox®). A high purity neurotoxic component, free of any complexing proteins, is for example available from Merz Pharmaceuticals GmbH, Frankfurt (Xeomin®).

Clostridial neurotoxins are usually injected into the affected muscle tissue, bringing the agent close to the neuro-muscular end plate, i.e. close to the cellular receptor mediating its uptake into the nerve cell controlling said affected muscle. Various degrees of neurotoxin spread have been observed. The neurotoxin spread is thought to depend on the injected amount and the particular neurotoxin preparation. It can result in adverse side effects such as paralysis in nearby muscle tissue, which can largely be avoided by reducing the injected doses to the therapeutically relevant level. Overdosing can also trigger the immune system to generate neutralizing antibodies that inactivate the neurotoxin preventing it from relieving the involuntary muscle activity.

Due to high toxicity, severe side effects and the possible development of immunity, there is a strong demand to produce the toxins with the highest possible purity and reproducibility and to obtain clostridial neurotoxins with tightly regulated biological activity upon administration to humans. So far, this aspect has not been solved satisfactorily.

In WO 2011/000929, it is discussed to replace the N-terminal proline of clostridial neurotoxins by a lysine. However, WO 2011/000929 does not discuss how such a replacement could be achieved. Furthermore, it is suggested to insert an oligolysine sequence into the N-terminus. However, it is not described, where and how to perform such insertion.

### OBJECTS OF THE INVENTION

It was an object of the invention to establish a reliable and accurate method for manufacturing and obtaining recombinant proteins, such as clostridial neurotoxins, harbouring an N-terminal lysine. In particular, a highly effective, i.e. near-complete cleavage of a precursor protein at a defined, exposed, N-terminal cleavage site, i.e. without accidental cleavage at other sites, is intended by the invention. Such a method and novel precursor proteins, such as clostridial neurotoxins, used in such methods would also serve to satisfy the great need for recombinant proteins, particularly recombinant pharmaceutical proteins, such as clostridial neurotoxins, harbouring an N-terminal lysine.

### SUMMARY OF THE INVENTION

As the methionyl-aminopeptidase (MAP) does not enzymatically excise the N-terminal fMet if the second amino acid residue is lysine, recombinant proteins with an N-terminal lysine are not obtainable so far. However, as lysine is a destabilising amino acid when present at the N-terminus, the generation of recombinant proteins with an N-terminal lysine might be advantageous, especially for pharmaceutical recombinant proteins that are potentially harmful and whose biological activity in the human body has therefore to be tightly regulated.

Furthermore, an N-terminal lysine residue allows for coupling via the free amino group.

Surprisingly it has been found that proteins with an N-terminal lysine, such as clostridial neurotoxins with an N-terminal lysine, can be obtained recombinantly after expression in recombinant host cells, by cloning a sequence encoding an N-terminal motif X-Lys, which can be recognised by an endoprotease specific for a lysine in P′1 position, into a gene encoding a parental protein, such as a clostridial neurotoxin, and by subsequent cleavage with an endoprotease specific for a lysine in P′1 position. Additionally, folded protein regions, which are not exposed, were surprisingly found not to be cleaved by an endoprotease specific for a lysine in P′1 position.

Thus, in one aspect, the present invention relates to a method for the generation of a recombinant protein with an N-terminal lysine comprising the step of causing or allowing contacting of a precursor protein, which comprises an N-terminal motif X-Lys-linker, wherein X is an endoprotease recognition sequence, and wherein said linker comprises at least three amino acid residues comprising (i) at least a second Lys residue and/or a Thr residue and (ii) at least one consecutive Gly residues, with an endoprotease specifically cleaving between X and Lys.

In another aspect, the present invention relates to a precursor protein, wherein said precursor protein comprises an N-terminal motif X-Lys-linker, wherein X is an endoprotease recognition sequence, and wherein said linker comprises at least three amino acid residues comprising (i) at least a second Lys residue and/or a Thr residue, and (ii) at least two consecutive Gly residues.

In another aspect, the present invention relates to a recombinant protein, wherein the N-terminus of said recombinant protein consists of the sequence Lys-linker, wherein said linker comprises at least three amino acid residues comprising (i) at least a second Lys residue and/or a Thr residue, and (ii) at least two consecutive Gly residues; particularly wherein said recombinant protein comprises at least 50 amino acid residues, particularly at least 100 amino acid residues, particularly at least 200 amino acid residues.

In another aspect, the present invention relates to a nucleic acid sequence encoding the precursor protein of the present invention, particularly wherein said nucleic acid has the sequence as found in any one of SEQ ID NOs: 7 to 9.

In another aspect, the present invention relates to a method for obtaining the nucleic acid of the present invention, comprising the step of inserting a nucleic acid sequence coding for an N-terminal motif X-Lys-linker into a nucleic acid sequence encoding a parental protein.

In another aspect, the present invention relates to a vector comprising the nucleic acid sequence of the present invention, or the nucleic acid obtainable by the method of the present invention.

In yet another aspect, the present invention relates to a recombinant host cell comprising the nucleic acid sequence of the present invention, the nucleic acid obtainable by the method of the present invention, or the vector of the present invention.

In another aspect, the present invention relates to a method for generating the precursor protein of the present invention, or the recombinant protein of the present invention, comprising the step of expressing the nucleic acid sequence of the present invention, the nucleic acid sequence obtainable by the method of the present invention, or the vector of the present invention in a recombinant host cell, or cultivating the recombinant host cell of the present invention under conditions that result in the expression of said nucleic acid sequence.

In another aspect, the present invention relates to a pharmaceutical composition comprising the recombinant protein of the present invention.

### DETAILED DESCRIPTION OF THE INVENTION

The present invention may be understood more readily by reference to the following detailed description of the invention and the examples included therein.

In one aspect, the present invention relates to a method for the generation of a recombinant protein with an N-terminal lysine comprising the step of causing or allowing contacting of a precursor protein, which comprises an N-terminal motif X-Lys-linker, wherein X is an endoprotease recognition sequence, and wherein said linker comprises at least three amino acid residues comprising (i) at least a second Lys residue and/or a Thr residue, and (ii) at least two consecutive Gly residues, with an endoprotease specifically cleaving between X and Lys.

In the context of the present invention, the term “causing . . . contacting of a precursor protein . . . with an endoprotease” refers to an active and/or direct step of bringing said protein and said endoprotease in contact, whereas the term “allowing contacting of a precursor protein . . . with an endoprotease” refers to an indirect step of establishing conditions in such a way that said protein and said endoprotease are getting in contact to each other.

In the context of the present invention, the term “endoprotease” or “endopeptidase” refers to proteases that break peptide bonds of non-terminal amino acids (i.e. within the polypeptide chain). As they do not attack terminal amino acids, endoproteases cannot break down peptides into monomers.

In the context of the present invention, the term “endoprotease specifically cleaving between X and Lys” refers to particular endoproteases that are able to cleave polypeptide sequences carrying a certain recognition sequence X followed by a lysine residue between said sequence X and the lysine residue, thus creating a polypeptide carrying an N-terminal lysine residue. In the past, such endoproteases have been widely used for the fragmentation of large proteins for mass spectrometry analyses (see, for example, EP 2 081 025; Taouatas, Lys-N: A versatile enzyme for proteomics, Utrecht 2000, ISBN: 978-90-393-5488-9; Nonaka et al., J. Biochem. 124 (1998) 157-162), i.e. for the simultaneous cleavage or proteins at many different locations in order to create a large variety of different protein fragments. The targeted use of such endoproteases for the specific cleavage at the N-terminus of a precursor protein only has not yet been described so far.

In the context of the present invention, the term “comprises” or “comprising” means “including, but not limited to”. The term is intended to be open-ended, to specify the presence of any stated features, elements integers, steps or components, but not to preclude the presence or addition of one or more other features, elements, integers, steps, components, or groups thereof. The term “comprising” thus includes the more restrictive terms “consisting of” and “consisting essentially of”.

The N-terminal motif X-Lys can be recognised and cleaved by an endoprotease specific for a lysine in P′1 position.

The linker downstream of said N-terminal lysine exposes the N-terminal motif X-Lys, enabling the endoprotease specific for a lysine in P′1 position to recognise and cleave at said lysine residue. Preferably, said endoprotease cannot cleave at lysine residues in folded, non-exposed protein regions.

In the context of the present invention, the term “precursor protein” refers to a protein harbouring the cleavage signal for the generation of a cleaved protein fragment with an N-terminal lysine.

In the context of the present invention, the term “recombinant protein” refers to a protein that is produced by using recombinant technologies, i.e. by genetically engineering a nucleic acid sequence encoding the recombinant protein followed by expression of said nucleic acid sequence in an appropriate in vitro or in vivo expression system. Thus, a recombinant protein is not produced by chemical protein synthesis. In particular embodiments, the term refers to a composition comprising a protein, that is obtained by expression of the protein in a heterologous cell such as E. coli, and including, but not limited to, the raw material obtained from a fermentation process (supernatant, composition after cell lysis), a fraction comprising a protein obtained from separating the ingredients of such a raw material in a purification process, an isolated and essentially pure protein, and a formulation for pharmaceutical and/or aesthetic use comprising a protein, such as a clostridial neurotoxin, and additionally pharmaceutically acceptable solvents and/or excipients.

In particular embodiments, cleavage of the precursor protein at an N-terminal motif X-Lys with an endoprotease specific for a lysine in P′1 position is near-complete.

In the context of the present invention, the term “P′1 position” refers to the amino acid position in a polypeptide chain directly after (i.e. C-terminally of) the cleavage site for a protease.

In the context of the present invention the term “near-complete” is defined as more than about 95% cleavage, particularly more than about 97.5%, more particularly more than about 99% as determined by SDS-PAGE and subsequent Western Blot or reversed phase chromatography.

Thus, in particular embodiments of the method of the present invention, the precursor protein is cleaved at the N-terminal motif X-Lys to more than about 97.5%, more particularly more than about 99% as determined by SDS-PAGE or reversed phase chromatography.

In the context of the present invention, the term “about” or “approximately” means within 20%, alternatively within 10%, including within 5% of a given value or range. Alternatively, especially in biological systems, the term “about” means within about a log (i.e. an order of magnitude), including within a factor of two of a given value.

In particular embodiments, cleavage of the precursor protein at the N-terminal motif X- Lys is without accidental cleavage at other internal lysine residues in non-exposed folded protein regions.

In the context of the present invention, the term “without accidental cleavage” means that less than about 10%, particularly less than about 1%, more particularly less than about 0.1% of cleavage products are cleavage products other than the desired recombinant protein with an N-terminal lysine resulting from cleavage of the precursor protein at the N-terminal motif X-Lys, as determined by liquid chromatography-mass spectrometry (LC-MS) or mass spectrometry.

Thus, in particular embodiments of the method of the present invention, less than about 10%, particularly less than about 1%, more particularly less than about 0.1% of cleavage products are cleavage products other than the desired recombinant protein with an N-terminal lysine resulting from cleavage of the precursor protein at the N-terminal motif X-Lys, as determined by LC-MS or mass spectrometry.

Thus, in particular embodiments of the method of the present invention, the precursor protein comprises a C-terminal part consisting of the sequence X-Lys-linker-P, wherein P is a parental protein sequence, and wherein the recombinant protein (i.e. after cleavage) consists of the sequence Lys-linker-P. In this context, the term “parental protein sequence” relates to a protein sequence that is intended to be modified by an N-terminal lysine residue.

In particular embodiments, the cleavage reaction is performed under conditions selected from the following: amount of endoprotease: between about 0.0005 and about 0.005 U per 1 μg precursor protein; reaction temperature between about 15° C. and about 25° C.; reaction time between about 1 h and about 3 h; buffer solution with pH between about 7 and about 8, and osmolarity between about 250 and about 500 mOsm.

In particular embodiments, the cleavage reaction is performed under the following conditions: 0.001 U Lys-N per 1 μg precursor protein; reaction temperature 20° C.; reaction time 2 h; pH 7.7; 20 mM Tris-HCl, 150 mM NaCl, 2.5 mM CaCl₂.

In particular embodiments, the cleavage reaction is performed with crude host cell lysates containing said precursor protein.

In other particular embodiments, the precursor protein is purified or partially purified, particularly by a first chromatographic enrichment step, prior to the cleavage reaction.

In the context of the present invention, the term “purified” relates to more than about 90% purity. In the context of the present invention, the term “partially purified” relates to purity of less than about 90% and an enrichment of more than about two fold.

In certain embodiments, the method of the present invention further comprises the step of obtaining a recombinant nucleic acid sequence encoding said precursor protein by the insertion of a nucleic acid sequence encoding said N-terminal motif X-Lys-linker into a nucleic acid sequence encoding a parental protein.

In the context of the present invention, the term “parental protein” refers to an initial protein that is generated under standard expression condition with an N-terminal residue different from lysine.

In a particular embodiment, a recombinant protein with an N-terminal lysine having a shortened duration of effectiveness compared to the parental protein is generated.

In particular embodiments, the method of the present invention further comprises the step of heterologously expressing a nucleic acid sequence encoding said precursor protein in a host cell before causing or allowing contacting of said precursor protein with said endoprotease.

In a particular embodiment, said endoprotease is Lys-N from Grifola frondosa, and is also known as GFMEP (Taouatas, loc. cit., p. 33). This zinc metalloendopeptidase consists of a single polypeptide chain with 167 amino acids residues and cleaves proteins on the amino side of lysine residues. Lys-N is commonly used for protein digestion in proteomics. It has been shown that a broad spectrum of lysine-containing sequences are cleaved by Lys-N (Nonaka et al., loc. cit., p. 159, Tables I and II. Surprisingly, the present inventors have found that it is possible to identify a sequence X-Lys-linker that results in a highly specific cleavage between X and the lysine residue, while leaving other lysine-containing sequence stretches intact, particularly under the reaction conditions described herein.

In a particular embodiment, Lys-N is recombinant Lys-N.

In particular embodiments, said endoprotease recognition sequence X has the sequence VRGIITS (SEQ ID NO: 10).

In particular embodiments, said linker has the sequence TKGₙ, wherein n is an integer larger than or equal to 1, particularly selected from the range of 2 to 12, particularly 2 to 8, particularly selected from 2, 4, and 8.

In another particular embodiment, said endoprotease is POMEP from Pleurotus ostreatus (Nonaka, loc. cit.; Dohmae et al., Biosci. Biotechnol. Biochem. 59 (1995) 2074-2080).

In certain embodiments, the parental protein is a clostridial neurotoxin.

In the context of the present invention, the term “clostridial neurotoxin” refers to a natural neurotoxin obtainable from bacteria of the class Clostridia, including Clostridium tetani and Clostridium botulinum, or to a neurotoxin obtainable from alternative sources, including from recombinant technologies or from genetic or chemical modification. Particularly, the clostridial neurotoxins have endopeptidase activity.

In a particular embodiment a recombinant clostridial neurotoxin with an N-terminal lysine exhibiting a shortened duration of effectiveness compared to the parental clostridial neurotoxin is generated.

In particular embodiments the clostridial neurotoxin is selected from a Clostridium botulinum neurotoxin serotype A, B, C, D, E, F, and G, or from a functional variant of such a Clostridium botulinum neurotoxin.

In the context of the present invention, the term “Clostridium botulinum neurotoxin serotype A, B, C, D, E, F, and G” refers to neurotoxins obtainable from Clostridium botulinum. Currently, seven serologically distinct types, designated serotypes A, B, C, D, E, F, and G are known, including certain subtypes (e.g. A1, A2, A3, A4 and A5).

In preferred embodiments the clostridial neurotoxin is selected from a Clostridium botulinum neurotoxin serotype A and E, particularly Clostridium botulinum neurotoxin serotype E, or from a functional variant of any such Clostridium botulinum neurotoxin.

In the context of the present invention, the term “functional variant of a Clostridium botulinum neurotoxin” refers to a neurotoxin that differs in the amino acid sequence and/or the nucleic acid sequence encoding the amino acid sequence from a Clostridium botulinum neurotoxin but is still functionally active. In this context “functionally active” or biologically active” means that said variant can bind to the neurotoxin receptor, is taken up into the nerve cell, and is capable of inhibiting neurotransmitter release from the affected nerve cell. In the context of the present invention, the term “functionally active” refers to the property of a recombinant clostridial neurotoxin to perform the biological functions of a naturally occurring Clostridium botulinum neurotoxin to at least about 50%, particularly to at least about 60%, to at least about 70%, to at least about 80%, and most particularly to at least about 90%, where the biological functions include, but are not limited to, entry of the neurotoxin into a neuronal cell, release of the light chain from the two-chain neurotoxin, and endopeptidase activity of the light chain.

On the protein level, a functional variant will maintain key features of the corresponding Clostridium botulinum neurotoxin, such as key residues for the endopeptidase activity in the light chain, or key residues for the attachment to the neurotoxin receptors or for translocation through the endosomal membrane in the heavy chain, but may contain one or more mutations comprising a deletion of one or more amino acids of the parental Clostridium botulinum neurotoxin, an addition of one or more amino acids of the parental Clostridium botulinum neurotoxin, and/or a substitution of one or more amino acids of the parental Clostridium botulinum neurotoxin. Preferably, said deleted, added and/or substituted amino acids are consecutive amino acids. According to the teaching of the present invention, any number of amino acids may be added, deleted, and/or substituted, as long as the functional variant remains biologically active. For example, 1, 2, 3, 4, 5, up to 10, up to 15, up to 25, up to 50, up to 100, up to 200, up to 400, up to 500 amino acids or even more amino acids may be added, deleted, and/or substituted. Accordingly, a functional variant of the neurotoxin may be a biologically active fragment of a naturally occurring neurotoxin. This neurotoxin fragment may contain an N-terminal, C-terminal, and/or one or more internal deletion(s).

In another embodiment, the functional variant of a clostridial neurotoxin additionally comprises a signal peptide. Usually said signal peptide will be located at the N-terminus of the neurotoxin. Many such signal peptides are known in the art and are comprised by the present invention. In particular, the signal peptide results in transport of the neurotoxin across a biological membrane, such as the membrane of the endoplasmic reticulum, the Golgi membrane or the plasma membrane of a eukaryotic or prokaryotic cell. It has been found that signal peptides, when attached to the neurotoxin, will mediate secretion of the neurotoxin into the supernatant of the cells. In certain embodiments, the signal peptide will be cleaved off in the course of, or subsequent to, secretion, so that the secreted protein lacks the N-terminal signal peptide, is composed of separate light and heavy chains, which are covalently linked by disulfide bridges, and is proteolytically active.

In particular embodiments, the functional variant has a sequence identity of at least about 40%, at least about 50%, at least about 60%, at least about 70% or most particularly at least about 80%, and a sequence homology of at least about 60%, at least about 70%, at least about 80%, at least about 90%, or most particularly at least about 95%. Methods and algorithms for determining sequence identity and/or homology, including the comparison of variants having deletions, additions, and/or substitutions relative to a parental sequence, are well known to the practitioner of ordinary skill in the art. On the DNA level, the nucleic acid sequences encoding the functional homologue and the parental Clostridium neurotoxin may differ to a larger extent due to the degeneracy of the genetic code. It is known that the usage of codons is different between prokaryotic and eukaryotic organisms. Thus, when expressing a prokaryotic protein such as a Clostridium neurotoxin, in a eukaryotic expression system, it may be necessary, or at least helpful, to adapt the nucleic acid sequence to the codon usage of the expression host cell, meaning that sequence identity or homology may be rather low on the nucleic acid level.

In the context of the present invention, the term “variant” refers to a neurotoxin that is a chemically, enzymatically, or genetically modified derivative of a parental Clostridium neurotoxin, including chemically or genetically modified neurotoxin from C. botulinum, particularly of C. botulinum neurotoxin serotype E. A chemically modified derivative may be one that is modified by pyruvation, phosphorylation, sulfatation, lipidation, pegylation, glycosylation and/or the chemical addition of an amino acid or a polypeptide comprising between 2 and about 100 amino acids, including modification occurring in the eukaryotic host cell used for expressing the derivative. An enzymatically modified derivative is one that is modified by the activity of enzymes, such as endo- or exoproteolytic enzymes, including by modification by enzymes of the eukaryotic host cell used for expressing the derivative. As pointed out above, a genetically modified derivative is one that has been modified by deletion or substitution of one or more amino acids contained in, or by addition of one or more amino acids (including polypeptides comprising between 2 and about 100 amino acids) to, the amino acid sequence of said Clostridium neurotoxin. Methods for designing and constructing such chemically or genetically modified derivatives and for testing of such variants for functionality are well known to anyone of ordinary skill in the art.

In particular embodiments, said clostridial neurotoxin is a functional variant of a clostridial neurotoxin selected from a Clostridium botulinum neurotoxin serotype A, B, C, D, E, F, and G, particularly serotype A or E, particularly E, wherein said functional variant comprises in the linker region between the neurotoxin light chain and the neurotoxin heavy chain a second copy of the endoprotease recognition sequence VRGIITS (SEQ ID NO: 10).

In certain embodiments, the precursor protein is expressed in E. coli host cells.

In certain embodiments, the E. coli cells are selected from E. coli XL1-Blue, Nova Blue, TOP10, XL10-Gold, BL21, and K12.

In another aspect, the present invention relates to a precursor protein, wherein said precursor protein comprises an N-terminal motif X-Lys-linker, wherein X is an endoprotease recognition sequence, and wherein said linker comprises at least three amino acid residues comprising (i) at least a second Lys residue and/or a Thr residue, and (ii) at least two consecutive Gly residues.

In a particular embodiment, the endoprotease recognition sequence X has the sequence VRGIITS (SEQ ID NO: 10).

In a particular embodiment, the linker has the sequence TKGₙ, wherein n is an integer larger than or equal to 1 particularly selected from the range of 2 to 12, particularly 2 to 8, particularly selected from 2, 4, and 8.

In a preferred embodiment, said precursor protein is a clostridial neurotoxin precursor.

In a preferred embodiment, the clostridial neurotoxin precursor has a sequence as found in any one of SEQ ID NOs: 1 to 3.

In another aspect, the present invention relates to a recombinant protein, wherein the N-terminus of said recombinant protein consists of the sequence Lys-linker, wherein said linker comprises at least three amino acid residues comprising (i) at least a second Lys residue and/or a Thr residue, and (ii) at least two consecutive Gly residues; particularly wherein said recombinant protein comprises at least 50 amino acid residues, particularly at least 100 amino acid residues, particularly at least 200 amino acid residues.

So far, only short peptides with such an N-terminus were known (see, for example, CN 1 724 566), which, however, are no recombinant proteins.

In particular embodiments, the linker has the sequence TKGₙ, wherein n is an integer larger than or equal to 2, particularly selected from the range of 2 to 12, particularly 2 to 8, particularly selected from 2, 4, and 8.

In particular embodiments, the recombinant protein is a clostridial neurotoxin.

In particular embodiments, the clostridial neurotoxin has a sequence as found in any one of SEQ ID NOs: 4 to 6.

In another aspect, the present invention relates to a nucleic acid sequence encoding a precursor protein of the present invention.

In particular embodiments, the nucleic acid sequence encodes a clostridial neurotoxin.

In particular such embodiments, said nucleic acid sequence has the sequence as found in any one of SEQ ID NOs: 7 to 9.

In another aspect, the present invention relates to a method for obtaining the nucleic acid sequence of the present invention, comprising the step of inserting a nucleic acid sequence coding for an N-terminal motif X-Lys-linker into a nucleic acid sequence encoding a parental protein.

In particular embodiments, the endoprotease recognition sequence X has the sequence VRGIITS (SEQ ID NO: 10).

In particular embodiments, the linker has the sequence TKGₙ, wherein n is an integer larger than or equal to 2, particularly selected from the range of 2 to 12, particularly 2 to 8, particularly selected from 2, 4, and 8.

In particular embodiments, the parental protein is a clostridial neurotoxin.

In another aspect, the present invention relates to a vector comprising the nucleic acid sequence of the present invention, or the nucleic acid obtainable by the method of the present invention.

In yet another aspect, the present invention relates to a recombinant host cell comprising the nucleic acid sequence of the present invention, the nucleic acid obtainable by the method of the present invention, or the vector of the present invention.

In particular embodiments, the E. coli cells are selected from E. coli XL1-Blue, Nova Blue, TOP10, XL10-Gold, BL21, and K12.

In another aspect, the present invention relates to a method for generating the precursor protein of the present invention, or the recombinant protein of the present invention, comprising the step of expressing the nucleic acid sequence of the present invention, the nucleic acid sequence obtainable by the method of the present invention, or the vector of the present invention in a recombinant host cell, or cultivating the recombinant host cell of the present invention under conditions that result in the expression of said nucleic acid sequence.

In particular embodiments, the precursor protein, or the recombinant protein, is purified after expression, or in the case of the recombinant protein, after the cleavage reaction. In particular such embodiments, the protein is purified by chromatography. In particular embodiments, the endoprotease is removed by immunoaffinity chromatography.

In another aspect, the present invention relates to a pharmaceutical composition comprising the recombinant protein of the present invention.

In particular embodiments, the recombinant protein is a clostridial neurotoxin.

In particular such embodiments, the pharmaceutical composition is for use in the treatment of a disease or condition taken from the list of: cervical dystonia (spasmodic torticollis), blepharospasm, severe primary axillary hyperhidrosis, achalasia, lower back pain, benign prostate hypertrophy, chronic focal painful neuropathies, migraine and other headache disorders, and cosmetic or aesthetic applications.

Additional indications where treatment with Botulinum neurotoxins is currently under investigation and where the pharmaceutical composition of the present invention may be used, include pediatric incontinence, incontinence due to overactive bladder, and incontinence due to neurogenic bladder, anal fissure, spastic disorders associated with injury or disease of the central nervous system including trauma, stroke, multiple sclerosis, Parkinson's disease, or cerebral palsy, focal dystonias affecting the limbs, face, jaw or vocal cords, temporomandibular joint (TMJ) pain disorders, diabetic neuropathy, wound healing, excessive salivation, vocal cord dysfunction, reduction of the Masseter muscle for decreasing the size of the lower jaw, treatment and prevention of chronic headache and chronic musculoskeletal pain, treatment of snoring noise, assistance in weight loss by increasing the gastric emptying time.

### EXAMPLES

### Example 1

### Generation of a Botulinum Toxin Mutant with an N-Terminal Cleavage Site for Lvs-N

A DNA Sequence coding for an endopeptidase recognition sequence, lysine and the required linker sequence (see Example 3) was added to the DNA sequence of botulinum toxin type E contained in an expression vector for E. coli via gene synthesis and subcloning. This construct was transformed into an E. coli expression strain (BL21) and the modified botulinum toxin was recombinantly expressed. Purification of the toxin from E. coli cell lysates was performed by affinity chromatography (his-tag) and a final size exclusion chromatography step.

### Example 2

### Cleavage with Lvs-N (Recombinant)

The purified botulinum toxin (example 1) was incubated with 0.001 U Lys-N per 1 μg toxin at pH 7.7 in 20 mM Tris-HCl, 150 mM NaCl, 2.5 mM CaCl₂ for 2 h at 20° C. In doing so, proteolytic cleavage N-terminally of exposed lysine residues occurs. Lysine residues present in folded protein regions, which are therefore not exposed, are not attacked. The successful proteolytic removal of the sequence N-terminal from the exposed lysine residue and thus the generation of an N-terminal lysine was analysed by immunoblotting for a tag, which is part of the N-terminal sequence, as well as by Edman degradation.

### Example 3

### Determination of N-Terminal Cleavage Motif

A series of BoNT/E-based constructs with N-terminal lysine containing motifs were constructed, and cleavage by Lys-N was tested as described in Example 2. The following Table 1 contains the results of these experiments.

TABLE 1

| | SEQ. | Cleavage |
|---|---|---|
| Sequence | ID NO: | by Lys-N? |
| M-K-GG-INS | 11 | NO |
| MA-YPYDVPDYA-K- | 12 | NO |
| GGGG-PKINS | | |
| MA-YPYDVPDYA-K- | 13 | NO |
| GGGG-K-GGGG-PKINS | | |
| MA-YPYDVPDYA- | 14 | YES |
| VRGIITS-KT-K-GGGG- | | |
| PKINS | | |
| MA-YPYDVPDYA- | 15 | YES |
| VRGIITS-KT-K- | | |
| GGGGGGGG-PKINS | | |
| MA-YPYDVPDYA- | 16 | NO |
| VRGIITS-K-GGGG-PKINS | | |
| MA-YPYDVPDYA- | 17 | NO |
| VRGIITS-K-PKINS | | |
| MA-YPYDVPDYA- | 18 | NO |
| VRGIITS-KT-PKINS | | |
| MA-YPYDVPDYA- | 19 | NO |
| VRGIITS-KT-K-PKINS | | |
| MA-YPYDVPDYA- | 20 | YES |
| VRGIITS-KT-K-GG-PKINS | | |

SEQ ID NO: 1
MAYPYDVPDYAVRGIITSKTKGGPKINSFNYNDPVNDRTILYIKPGGCQEFYKSFNIMKNIWIIPERN
VIGTTPQDFHPPTSLKNGDSSYYDPNYLQSDEEKDRFLKIVTKIFNRINNNLSGGILLEELSKANPYL
GNDNTPDNQFHIGDASAVEIKFSNGSQDILLPNVIIMGAEPDLFETNSSNISLRNNYMPSNHGFGSIA
IVTFSPEYSFRFNDNSMNEFIQDPALTLMHELIHSLHGLYGAKGITTKYTITQKQNPLITNIRGTNIE
EFLTFGGTDLNIITSAQSNDIYTNLLADYKKIASKLSKVQVSNPLLNPYKDVFEAKYGLDKDASGIYS
VNINKFNDIFKKLYSFTEFDLATKFQVKCRQTYIGQYKYFKLSNLLNDSIYNISEGYNINNLKVNFRG
QNANLNPRIITPITGRGLVKKIIRFCVRGIITSKTKSLVPRGSKALNDLCIEINNGELFFVASENSYN
DDNINTPKEIDDTVTSNNNYENDLDQVILNFNSESAPGLSDEKLNLTIQNDAYIPKYDSNGTSDIEQH
DVNELNVFFYLDAQKVPEGENNVNLTSSIDTALLEQPKIYTFFSSEFINNVNKPVQAALFVSWIQQVL
VDFTTEANQKSTVDKIADISIVVPYIGLALNIGNEAQKGNFKDALELLGAGILLEFEPELLIPTILVF
TIKSFLGSSDNKNKVIKAINNALKERDEKWKEVYSFIVSNWMTKINTQFNKRKEQMYQALQNQVNAIK
TIIESKYNSYTLEEKNELTNKYDIKQIENELNQKVSIAMNNIDRFLTESSISYLMKLINEVKINKLRE
YDENVKTYLLNYIIQHGSILGESQQELNSMVTDTLNNSIPFKLSSYTDDKILISYFNKFFKRIKSSSV
LNMRYKNDKYVDTSGYDSNININGDVYKYPTNKNQFGIYNDKLSEVNISQNDYIIYDNKYKNFSISFW
VRIPNYDNKIVNVNNEYTIINCMRDNNSGWKVSLNHNEIIWTLQDNAGINQKLAFNYGNANGISDYIN
KWIFVTITNDRLGDSKLYINGNLIDQKSILNLGNIHVSDNILFKIVNCSYTRYIGIRYFNIFDKELDE
TEIQTLYSNEPNTNILKDFWGNYLLYDKEYYLLNVLKPNNFIDRRKDSTLSINNIRSTILLANRLYSG
IKVKIQRVNNSSTNDNLVRKNDQVYINFVASKTHLFPLYADTATTNKEKTIKISSSGNRFNQVVVMNS
VGNNCTMNFKNNNGNNIGLLGFKADTVVASTWYYTHMRDHTNSNGCFWNFISEEHGWQEK
SEQ ID NO: 2
MAYPYDVPDYAVRGIITSKTKGGGGPKINSFNYNDPVNDRTILYIKPGGCQEFYKSFNIMKNIWIIPE
RNVIGTTPQDFHPPTSLKNGDSSYYDPNYLQSDEEKDRFLKIVTKIFNRINNNLSGGILLEELSKANP
YLGNDNTPDNQFHIGDASAVEIKFSNGSQDILLPNVIIMGAEPDLFETNSSNISLRNNYMPSNHGFGS
IAIVTFSPEYSFRFNDNSMNEFIQDPALTLMHELIHSLHGLYGAKGITTKYTITQKQNPLITNIRGTN
IEEFLTFGGTDLNIITSAQSNDIYTNLLADYKKIASKLSKVQVSNPLLNPYKDVFEAKYGLDKDASGI
YSVNINKFNDIFKKLYSFTEFDLATKFQVKCRQTYIGQYKYFKLSNLLNDSIYNISEGYNINNLKVNF
RGQNANLNPRIITPITGRGLVKKIIRFCVRGIITSKTKSLVPRGSKALNDLCIEINNGELFFVASENS
YNDDNINTPKEIDDTVTSNNNYENDLDQVILNFNSESAPGLSDEKLNLTIQNDAYIPKYDSNGTSDIE
QHDVNELNVFFYLDAQKVPEGENNVNLTSSIDTALLEQPKIYTFFSSEFINNVNKPVQAALFVSWIQQ
VLVDFTTEANQKSTVDKIADISIVVPYIGLALNIGNEAQKGNFKDALELLGAGILLEFEPELLIPTIL
VFTIKSFLGSSDNKNKVIKAINNALKERDEKWKEVYSFIVSNWMTKINTQFNKRKEQMYQALQNQVNA
IKTIIESKYNSYTLEEKNELTNKYDIKQIENELNQKVSIAMNNIDRFLTESSISYLMKLINEVKINKL
REYDENVKTYLLNYIIQHGSILGESQQELNSMVTDTLNNSIPFKLSSYTDDKILISYFNKFFKRIKSS
SVLNMRYKNDKYVDTSGYDSNININGDVYKYPTNKNQFGIYNDKLSEVNISQNDYIIYDNKYKNFSIS
FWVRIPNYDNKIVNVNNEYTIINCMRDNNSGWKVSLNHNEIIWTLQDNAGINQKLAFNYGNANGISDY
INKWIFVTITNDRLGDSKLYINGNLIDQKSILNLGNIHVSDNILFKIVNCSYTRYIGIRYFNIFDKEL
DETEIQTLYSNEPNTNILKDFWGNYLLYDKEYYLLNVLKPNNFIDRRKDSTLSINNIRSTILLANRLY
SGIKVKIQRVNNSSTNDNLVRKNDQVYINFVASKTHLFPLYADTATTNKEKTIKISSSGNRFNQVVVM
NSVGNNCTMNFKNNNGNNIGLLGFKADTVVASTWYYTHMRDHTNSNGCFWNFISEEHGWQEK
SEQ ID NO: 3
MAYPYDVPDYAVRGIITSKTKGGGGGGGGPKINSFNYNDPVNDRTILYIKPGGCQEFYKSFNIMKNIW
IIPERNVIGTTPQDFHPPTSLKNGDSSYYDPNYLQSDEEKDRFLKIVTKIFNRINNNLSGGILLEELS
KANPYLGNDNTPDNQFHIGDASAVEIKFSNGSQDILLPNVIIMGAEPDLFETNSSNISLRNNYMPSNH
GFGSIAIVTFSPEYSFRFNDNSMNEFIQDPALTLMHELIHSLHGLYGAKGITTKYTITQKQNPLITNI
RGTNIEEFLTFGGTDLNIITSAQSNDIYTNLLADYKKIASKLSKVQVSNPLLNPYKDVFEAKYGLDKD
ASGIYSVNINKFNDIFKKLYSFTEFDLATKFQVKCRQTYIGQYKYFKLSNLLNDSIYNISEGYNINNL
KVNFRGQNANLNPRIITPITGRGLVKKIIRFCVRGIITSKTKSLVPRGSKALNDLCIEINNGELFFVA
SENSYNDDNINTPKEIDDTVTSNNNYENDLDQVILNFNSESAPGLSDEKLNLTIQNDAYIPKYDSNGT
SDIEQHDVNELNVFFYLDAQKVPEGENNVNLTSSIDTALLEQPKIYTFFSSEFINNVNKPVQAALFVS
WIQQVLVDFTTEANQKSTVDKIADISIVVPYIGLALNIGNEAQKGNFKDALELLGAGILLEFEPELLI
PTILVFTIKSFLGSSDNKNKVIKAINNALKERDEKWKEVYSFIVSNWMTKINTQFNKRKEQMYQALQN
QVNAIKTIIESKYNSYTLEEKNELTNKYDIKQIENELNQKVSIAMNNIDRFLTESSISYLMKLINEVK
INKLREYDENVKTYLLNYIIQHGSILGESQQELNSMVTDTLNNSIPFKLSSYTDDKILISYFNKFFKR
IKSSSVLNMRYKNDKYVDTSGYDSNININGDVYKYPTNKNQFGIYNDKLSEVNISQNDYIIYDNKYKN
FSISFWVRIPNYDNKIVNVNNEYTIINCMRDNNSGWKVSLNHNEIIWTLQDNAGINQKLAFNYGNANG
ISDYINKWIFVTITNDRLGDSKLYINGNLIDQKSILNLGNIHVSDNILFKIVNCSYTRYIGIRYFNIF
DKELDETEIQTLYSNEPNTNILKDFWGNYLLYDKEYYLLNVLKPNNFIDRRKDSTLSINNIRSTILLA
NRLYSGIKVKIQRVNNSSTNDNLVRKNDQVYINFVASKTHLFPLYADTATTNKEKTIKISSSGNRFNQ
VVVMNSVGNNCTMNFKNNNGNNIGLLGFKADTVVASTWYYTHMRDHTNSNGCFWNFISEEHGWQEK
SEQ ID NO: 4
KTKGGPKINSFNYNDPVNDRTILYIKPGGCQEFYKSFNIMKNIWIIPERNVIGTTPQDFHPPTSLKNG
DSSYYDPNYLQSDEEKDRFLKIVTKIFNRINNNLSGGILLEELSKANPYLGNDNTPDNQFHIGDASAV
EIKFSNGSQDILLPNVIIMGAEPDLFETNSSNISLRNNYMPSNHGFGSIAIVTFSPEYSFRFNDNSMN
EFIQDPALTLMHELIHSLHGLYGAKGITTKYTITQKQNPLITNIRGTNIEEFLTFGGTDLNIITSAQS
NDIYTNLLADYKKIASKLSKVQVSNPLLNPYKDVFEAKYGLDKDASGIYSVNINKFNDIFKKLYSFTE
FDLATKFQVKCRQTYIGQYKYFKLSNLLNDSIYNISEGYNINNLKVNFRGQNANLNPRIITPITGRGL
VKKIIRFCVRGIITSKTKSLVPRGSKALNDLCIEINNGELFFVASENSYNDDNINTPKEIDDTVTSNN
NYENDLDQVILNFNSESAPGLSDEKLNLTIQNDAYIPKYDSNGTSDIEQHDVNELNVFFYLDAQKVPE
GENNVNLTSSIDTALLEQPKIYTFFSSEFINNVNKPVQAALFVSWIQQVLVDFTTEANQKSTVDKIAD
ISIVVPYIGLALNIGNEAQKGNFKDALELLGAGILLEFEPELLIPTILVFTIKSFLGSSDNKNKVIKA
INNALKERDEKWKEVYSFIVSNWMTKINTQFNKRKEQMYQALQNQVNAIKTIIESKYNSYTLEEKNEL
TNKYDIKQIENELNQKVSIAMNNIDRFLTESSISYLMKLINEVKINKLREYDENVKTYLLNYIIQHGS
ILGESQQELNSMVTDTLNNSIPFKLSSYTDDKILISYFNKFFKRIKSSSVLNMRYKNDKYVDTSGYDS
NININGDVYKYPTNKNQFGIYNDKLSEVNISQNDYIIYDNKYKNFSISFWVRIPNYDNKIVNVNNEYT
IINCMRDNNSGWKVSLNHNEIIWTLQDNAGINQKLAFNYGNANGISDYINKWIFVTITNDRLGDSKLY
INGNLIDQKSILNLGNIHVSDNILFKIVNCSYTRYIGIRYFNIFDKELDETEIQTLYSNEPNTNILKD
FWGNYLLYDKEYYLLNVLKPNNFIDRRKDSTLSINNIRSTILLANRLYSGIKVKIQRVNNSSTNDNLV
RKNDQVYINFVASKTHLFPLYADTATTNKEKTIKISSSGNRFNQVVVMNSVGNNCTMNFKNNNGNNIG
LLGFKADTVVASTWYYTHMRDHTNSNGCFWNFISEEHGWQEK
SEQ ID NO: 5
KTKGGGGPKINSFNYNDPVNDRTILYIKPGGCQEFYKSFNIMKNIWIIPERNVIGTTPQDFHPPTSLK
NGDSSYYDPNYLQSDEEKDRFLKIVTKIFNRINNNLSGGILLEELSKANPYLGNDNTPDNQFHIGDAS
AVEIKFSNGSQDILLPNVIIMGAEPDLFETNSSNISLRNNYMPSNHGFGSIAIVTFSPEYSFRFNDNS
MNEFIQDPALTLMHELIHSLHGLYGAKGITTKYTITQKQNPLITNIRGTNIEEFLTFGGTDLNIITSA
QSNDIYTNLLADYKKIASKLSKVQVSNPLLNPYKDVFEAKYGLDKDASGIYSVNINKFNDIFKKLYSF
TEFDLATKFQVKCRQTYIGQYKYFKLSNLLNDSIYNISEGYNINNLKVNFRGQNANLNPRIITPITGR
GLVKKIIRFCVRGIITSKTKSLVPRGSKALNDLCIEINNGELFFVASENSYNDDNINTPKEIDDTVTS
NNNYENDLDQVILNFNSESAPGLSDEKLNLTIQNDAYIPKYDSNGTSDIEQHDVNELNVFFYLDAQKV
PEGENNVNLTSSIDTALLEQPKIYTFFSSEFINNVNKPVQAALFVSWIQQVLVDFTTEANQKSTVDKI
ADISIVVPYIGLALNIGNEAQKGNFKDALELLGAGILLEFEPELLIPTILVFTIKSFLGSSDNKNKVI
KAINNALKERDEKWKEVYSFIVSNWMTKINTQFNKRKEQMYQALQNQVNAIKTIIESKYNSYTLEEKN
ELTNKYDIKQIENELNQKVSIAMNNIDRFLTESSISYLMKLINEVKINKLREYDENVKTYLLNYIIQH
GSILGESQQELNSMVTDTLNNSIPFKLSSYTDDKILISYFNKFFKRIKSSSVLNMRYKNDKYVDTSGY
DSNININGDVYKYPTNKNQFGIYNDKLSEVNISQNDYIIYDNKYKNFSISFWVRIPNYDNKIVNVNNE
YTIINCMRDNNSGWKVSLNHNEIIWTLQDNAGINQKLAFNYGNANGISDYINKWIFVTITNDRLGDSK
LYINGNLIDQKSILNLGNIHVSDNILFKIVNCSYTRYIGIRYFNIFDKELDETEIQTLYSNEPNTNIL
KDFWGNYLLYDKEYYLLNVLKPNNFIDRRKDSTLSINNIRSTILLANRLYSGIKVKIQRVNNSSTNDN
LVRKNDQVYINFVASKTHLFPLYADTATTNKEKTIKISSSGNRFNQVVVMNSVGNNCTMNFKNNNGNN
IGLLGFKADTVVASTWYYTHMRDHTNSNGCFWNFISEEHGWQEK
SEQ ID NO: 6
KTKGGGGGGGGPKINSFNYNDPVNDRTILYIKPGGCQEFYKSFNIMKNIWIIPERNVIGTTPQDFHPP
TSLKNGDSSYYDPNYLQSDEEKDRFLKIVTKIFNRINNNLSGGILLEELSKANPYLGNDNTPDNQFHI
GDASAVEIKFSNGSQDILLPNVIIMGAEPDLFETNSSNISLRNNYMPSNHGFGSIAIVTFSPEYSFRF
NDNSMNEFIQDPALTLMHELIHSLHGLYGAKGITTKYTITQKQNPLITNIRGTNIEEFLTFGGTDLNI
ITSAQSNDIYTNLLADYKKIASKLSKVQVSNPLLNPYKDVFEAKYGLDKDASGIYSVNINKFNDIFKK
LYSFTEFDLATKFQVKCRQTYIGQYKYFKLSNLLNDSIYNISEGYNINNLKVNFRGQNANLNPRIITP
ITGRGLVKKIIRFCVRGIITSKTKSLVPRGSKALNDLCIEINNGELFFVASENSYNDDNINTPKEIDD
TVTSNNNYENDLDQVILNFNSESAPGLSDEKLNLTIQNDAYIPKYDSNGTSDIEQHDVNELNVFFYLD
AQKVPEGENNVNLTSSIDTALLEQPKIYTFFSSEFINNVNKPVQAALFVSWIQQVLVDFTTEANQKST
VDKIADISIVVPYIGLALNIGNEAQKGNFKDALELLGAGILLEFEPELLIPTILVFTIKSFLGSSDNK
NKVIKAINNALKERDEKWKEVYSFIVSNWMTKINTQFNKRKEQMYQALQNQVNAIKTIIESKYNSYTL
EEKNELTNKYDIKQIENELNQKVSIAMNNIDRFLTESSISYLMKLINEVKINKLREYDENVKTYLLNY
IIQHGSILGESQQELNSMVTDTLNNSIPFKLSSYTDDKILISYFNKFFKRIKSSSVLNMRYKNDKYVD
TSGYDSNININGDVYKYPTNKNQFGIYNDKLSEVNISQNDYIIYDNKYKNFSISFWVRIPNYDNKIVN
VNNEYTIINCMRDNNSGWKVSLNHNEIIWTLQDNAGINQKLAFNYGNANGISDYINKWIFVTITNDRL
GDSKLYINGNLIDQKSILNLGNIHVSDNILFKIVNCSYTRYIGIRYFNIFDKELDETEIQTLYSNEPN
TNILKDFWGNYLLYDKEYYLLNVLKPNNFIDRRKDSTLSINNIRSTILLANRLYSGIKVKIQRVNNSS
TNDNLVRKNDQVYINFVASKTHLFPLYADTATTNKEKTIKISSSGNRFNQVVVMNSVGNNCTMNFKNN
NGNNIGLLGFKADTVVASTWYYTHMRDHTNSNGCFWNFISEEHGWQEK
SEQ ID NO: 7
ATGGCATATCCGTATGATGTTCCGGATTATGCAGTTCGTGGTATTATTACCAGCAAAACCAAAGGTGG
CCCGAAAATCAACAGCTTCAACTATAACGATCCGGTGAACGATCGTACCATCCTGTATATTAAACCGG
GCGGTTGCCAGGAATTTTACAAAAGCTTCAACATCATGAAAAACATCTGGATTATTCCGGAACGTAAC
GTGATTGGCACCACCCCGCAGGATTTTCATCCGCCGACCAGCCTGAAAAACGGCGATAGCAGCTATTA
TGATCCGAACTATCTGCAGTCTGATGAAGAAAAAGATCGCTTCCTGAAAATCGTGACCAAAATCTTCA
ACCGCATCAACAACAACCTGAGCGGCGGCATTCTGCTGGAAGAACTGAGCAAAGCGAATCCGTATCTG
GGCAACGATAACACTCCAGATAACCAGTTTCATATTGGTGATGCGAGCGCGGTGGAAATTAAATTTAG
CAACGGCTCTCAGGACATTCTGCTGCCGAACGTGATTATTATGGGCGCGGAACCGGACCTGTTTGAAA
CCAACAGCAGCAACATTAGCCTGCGTAACAACTATATGCCGAGCAACCATGGTTTTGGCAGCATTGCG
ATTGTGACCTTTAGCCCGGAATATAGCTTTCGCTTCAACGATAACAGCATGAACGAATTTATTCAGGA
CCCGGCGCTGACCCTGATGCACGAGCTGATTCATAGCCTGCATGGCCTGTATGGCGCGAAAGGCATTA
CCACCAAATATACCATCACCCAGAAACAGAATCCGCTGATTACCAACATTCGTGGCACCAACATTGAA
GAATTTCTGACCTTTGGCGGCACCGATCTGAACATTATTACCAGCGCGCAGAGCAACGATATCTATAC
CAACCTGCTGGCCGATTATAAAAAAATCGCGTCTAAACTGAGCAAAGTGCAGGTGAGCAATCCGCTGC
TGAATCCGTATAAAGATGTGTTTGAAGCGAAATATGGCCTGGATAAAGATGCTAGCGGCATTTATAGC
GTGAACATCAACAAATTCAACGACATCTTCAAAAAACTGTATAGCTTTACCGAATTTGATCTGGCCAC
CAAATTTCAGGTGAAATGCCGCCAGACCTATATTGGCCAGTATAAATATTTTAAACTGAGCAACCTGC
TGAACGATAGCATTTACAACATCAGCGAAGGCTATAACATCAACAACCTGAAAGTGAACTTTCGTGGC
CAGAACGCGAATTTAAATCCGCGTATTATTACCCCGATTACCGGCCGTGGACTAGTGAAAAAAATTAT
CCGTTTTTGCGTGCGTGGCATTATCACCAGCAAAACCAAAAGCCTGGTGCCGCGTGGCAGCAAAGCGT
TAAATGATTTATGCATCGAAATCAACAACGGCGAACTGTTTTTTGTGGCGAGCGAAAACAGCTATAAC
GATGATAACATCAACACCCCGAAAGAAATTGATGATACCGTGACCAGCAATAACAACTACGAAAACGA
TCTGGATCAGGTGATTCTGAACTTTAACAGCGAAAGCGCACCGGGCCTGTCTGATGAAAAACTGAACC
TGACCATTCAGAACGATGCGTATATCCCGAAATATGATAGCAACGGCACCAGCGATATTGAACAGCAT
GATGTGAACGAACTGAACGTGTTTTTTTATCTGGATGCGCAGAAAGTGCCGGAAGGCGAAAACAACGT
GAATCTGACCAGCTCAATTGATACCGCGCTGCTGGAACAGCCGAAAATCTATACCTTTTTTAGCAGCG
AATTCATCAACAACGTGAACAAACCGGTGCAGGCGGCGCTGTTTGTGAGCTGGATTCAGCAGGTGCTG
GTTGATTTTACCACCGAAGCGAACCAGAAAAGCACCGTGGATAAAATTGCGGATATTAGCATTGTGGT
GCCGTATATTGGCCTGGCCCTGAACATTGGCAACGAAGCGCAGAAAGGCAACTTTAAAGATGCGCTGG
AACTGCTGGGTGCGGGCATTCTGCTGGAATTTGAACCGGAACTGCTGATTCCGACCATTCTGGTGTTT
ACCATCAAAAGCTTTCTGGGCAGCAGCGATAACAAAAACAAAGTGATCAAAGCGATTAACAACGCGCT
GAAAGAACGTGATGAAAAATGGAAAGAAGTGTATAGCTTCATTGTGTCTAACTGGATGACCAAAATCA
ACACCCAGTTCAACAAACGTAAAGAACAAATGTATCAGGCGCTGCAGAACCAGGTGAACGCGATTAAA
ACCATCATCGAAAGCAAATACAACAGCTACACCCTGGAAGAAAAAAACGAACTGACCAACAAATATGA
CATCAAACAAATCGAAAATGAACTGAACCAGAAAGTGAGCATTGCCATGAACAACATTGATCGCTTTC
TGACCGAAAGCAGCATTAGCTACCTGATGAAACTGATCAACGAAGTGAAAATCAACAAACTGCGCGAA
TATGATGAAAACGTGAAAACCTACCTGCTGAACTATATTATTCAGCATGGCAGCATTCTGGGCGAAAG
CCAGCAAGAACTGAACAGCATGGTTACCGATACCCTGAACAACAGCATTCCGTTTAAACTGAGCAGCT
ACACCGATGATAAAATCCTGATCAGCTACTTCAACAAATTCTTCAAACGCATCAAAAGCAGCAGCGTG
CTGAACATGCGTTATAAAAACGATAAATACGTAGATACCAGCGGCTATGATAGCAATATCAACATTAA
CGGTGATGTGTATAAATACCCGACCAACAAAAACCAGTTCGGCATCTACAACGATAAACTGAGCGAAG
TGAACATTAGCCAGAACGATTATATCATCTACGATAATAAATATAAAAACTTCAGCATCAGCTTTTGG
GTGCGTATTCCGAACTACGATAACAAAATCGTGAACGTGAACAACGAATACACCATCATTAACTGCAT
GCGTGATAACAACAGCGGCTGGAAAGTGAGCCTGAACCATAACGAAATCATCTGGACCCTGCAGGATA
ACGCCGGCATTAACCAGAAACTGGCCTTTAACTATGGCAACGCGAACGGCATTAGCGATTACATCAAC
AAATGGATCTTTGTGACCATTACCAACGATCGTCTGGGCGATAGCAAACTGTATATTAACGGCAACCT
GATCGACCAGAAAAGCATTCTGAACCTGGGCAACATTCATGTGAGCGATAACATCCTGTTCAAAATTG
TGAACTGCAGCTATACCCGTTATATTGGCATCCGCTATTTCAACATCTTCGATAAAGAACTGGATGAA
ACCGAAATTCAGACCCTGTATAGCAACGAACCGAACACCAACATCCTGAAAGATTTCTGGGGCAACTA
TCTGCTGTACGATAAAGAATATTATCTGCTGAACGTGCTGAAACCGAACAACTTTATTGATCGCCGTA
AAGATAGCACCCTGAGCATTAACAACATTCGTAGCACCATTCTGCTGGCCAACCGTCTGTATAGCGGC
ATTAAAGTGAAAATTCAGCGCGTGAACAATAGCAGCACCAACGATAACCTGGTGCGTAAAAACGATCA
GGTGTATATCAACTTTGTGGCCAGCAAAACCCACCTGTTTCCGCTGTATGCGGATACCGCGACCACCA
ACAAAGAAAAAACCATTAAAATCAGCAGCAGCGGCAACCGTTTTAACCAGGTGGTGGTGATGAACAGC
GTGGGCAACAACTGTACAATGAACTTCAAAAACAACAACGGCAACAACATTGGCCTGCTGGGCTTTAA
AGCGGATACCGTGGTGGCGAGCACCTGGTATTATACCCACATGCGTGATCATACCAACAGCAACGGCT
GCTTTTGGAACTTTATTAGCGAAGAACATGGCTGGCAGGAAAAATGA
SEQ ID NO: 8
ATGGCATATCCGTATGATGTTCCGGATTATGCAGTTCGTGGTATTATTACCAGCAAAACCAAAGGTGG
TGGCGGCCCGAAAATCAACAGCTTCAACTATAACGATCCGGTGAACGATCGTACCATCCTGTATATTA
AACCGGGCGGTTGCCAGGAATTTTACAAAAGCTTCAACATCATGAAAAACATCTGGATTATTCCGGAA
CGTAACGTGATTGGCACCACCCCGCAGGATTTTCATCCGCCGACCAGCCTGAAAAACGGCGATAGCAG
CTATTATGATCCGAACTATCTGCAGTCTGATGAAGAAAAAGATCGCTTCCTGAAAATCGTGACCAAAA
TCTTCAACCGCATCAACAACAACCTGAGCGGCGGCATTCTGCTGGAAGAACTGAGCAAAGCGAATCCG
TATCTGGGCAACGATAACACTCCAGATAACCAGTTTCATATTGGTGATGCGAGCGCGGTGGAAATTAA
ATTTAGCAACGGCTCTCAGGACATTCTGCTGCCGAACGTGATTATTATGGGCGCGGAACCGGACCTGT
TTGAAACCAACAGCAGCAACATTAGCCTGCGTAACAACTATATGCCGAGCAACCATGGTTTTGGCAGC
ATTGCGATTGTGACCTTTAGCCCGGAATATAGCTTTCGCTTCAACGATAACAGCATGAACGAATTTAT
TCAGGACCCGGCGCTGACCCTGATGCACGAGCTGATTCATAGCCTGCATGGCCTGTATGGCGCGAAAG
GCATTACCACCAAATATACCATCACCCAGAAACAGAATCCGCTGATTACCAACATTCGTGGCACCAAC
ATTGAAGAATTTCTGACCTTTGGCGGCACCGATCTGAACATTATTACCAGCGCGCAGAGCAACGATAT
CTATACCAACCTGCTGGCCGATTATAAAAAAATCGCGTCTAAACTGAGCAAAGTGCAGGTGAGCAATC
CGCTGCTGAATCCGTATAAAGATGTGTTTGAAGCGAAATATGGCCTGGATAAAGATGCTAGCGGCATT
TATAGCGTGAACATCAACAAATTCAACGACATCTTCAAAAAACTGTATAGCTTTACCGAATTTGATCT
GGCCACCAAATTTCAGGTGAAATGCCGCCAGACCTATATTGGCCAGTATAAATATTTTAAACTGAGCA
ACCTGCTGAACGATAGCATTTACAACATCAGCGAAGGCTATAACATCAACAACCTGAAAGTGAACTTT
CGTGGCCAGAACGCGAATTTAAATCCGCGTATTATTACCCCGATTACCGGCCGTGGACTAGTGAAAAA
AATTATCCGTTTTTGCGTGCGTGGCATTATCACCAGCAAAACCAAAAGCCTGGTGCCGCGTGGCAGCA
AAGCGTTAAATGATTTATGCATCGAAATCAACAACGGCGAACTGTTTTTTGTGGCGAGCGAAAACAGC
TATAACGATGATAACATCAACACCCCGAAAGAAATTGATGATACCGTGACCAGCAATAACAACTACGA
AAACGATCTGGATCAGGTGATTCTGAACTTTAACAGCGAAAGCGCACCGGGCCTGTCTGATGAAAAAC
TGAACCTGACCATTCAGAACGATGCGTATATCCCGAAATATGATAGCAACGGCACCAGCGATATTGAA
CAGCATGATGTGAACGAACTGAACGTGTTTTTTTATCTGGATGCGCAGAAAGTGCCGGAAGGCGAAAA
CAACGTGAATCTGACCAGCTCAATTGATACCGCGCTGCTGGAACAGCCGAAAATCTATACCTTTTTTA
GCAGCGAATTCATCAACAACGTGAACAAACCGGTGCAGGCGGCGCTGTTTGTGAGCTGGATTCAGCAG
GTGCTGGTTGATTTTACCACCGAAGCGAACCAGAAAAGCACCGTGGATAAAATTGCGGATATTAGCAT
TGTGGTGCCGTATATTGGCCTGGCCCTGAACATTGGCAACGAAGCGCAGAAAGGCAACTTTAAAGATG
CGCTGGAACTGCTGGGTGCGGGCATTCTGCTGGAATTTGAACCGGAACTGCTGATTCCGACCATTCTG
GTGTTTACCATCAAAAGCTTTCTGGGCAGCAGCGATAACAAAAACAAAGTGATCAAAGCGATTAACAA
CGCGCTGAAAGAACGTGATGAAAAATGGAAAGAAGTGTATAGCTTCATTGTGTCTAACTGGATGACCA
AAATCAACACCCAGTTCAACAAACGTAAAGAACAAATGTATCAGGCGCTGCAGAACCAGGTGAACGCG
ATTAAAACCATCATCGAAAGCAAATACAACAGCTACACCCTGGAAGAAAAAAACGAACTGACCAACAA
ATATGACATCAAACAAATCGAAAATGAACTGAACCAGAAAGTGAGCATTGCCATGAACAACATTGATC
GCTTTCTGACCGAAAGCAGCATTAGCTACCTGATGAAACTGATCAACGAAGTGAAAATCAACAAACTG
CGCGAATATGATGAAAACGTGAAAACCTACCTGCTGAACTATATTATTCAGCATGGCAGCATTCTGGG
CGAAAGCCAGCAAGAACTGAACAGCATGGTTACCGATACCCTGAACAACAGCATTCCGTTTAAACTGA
GCAGCTACACCGATGATAAAATCCTGATCAGCTACTTCAACAAATTCTTCAAACGCATCAAAAGCAGC
AGCGTGCTGAACATGCGTTATAAAAACGATAAATACGTAGATACCAGCGGCTATGATAGCAATATCAA
CATTAACGGTGATGTGTATAAATACCCGACCAACAAAAACCAGTTCGGCATCTACAACGATAAACTGA
GCGAAGTGAACATTAGCCAGAACGATTATATCATCTACGATAATAAATATAAAAACTTCAGCATCAGC
TTTTGGGTGCGTATTCCGAACTACGATAACAAAATCGTGAACGTGAACAACGAATACACCATCATTAA
CTGCATGCGTGATAACAACAGCGGCTGGAAAGTGAGCCTGAACCATAACGAAATCATCTGGACCCTGC
AGGATAACGCCGGCATTAACCAGAAACTGGCCTTTAACTATGGCAACGCGAACGGCATTAGCGATTAC
ATCAACAAATGGATCTTTGTGACCATTACCAACGATCGTCTGGGCGATAGCAAACTGTATATTAACGG
CAACCTGATCGACCAGAAAAGCATTCTGAACCTGGGCAACATTCATGTGAGCGATAACATCCTGTTCA
AAATTGTGAACTGCAGCTATACCCGTTATATTGGCATCCGCTATTTCAACATCTTCGATAAAGAACTG
GATGAAACCGAAATTCAGACCCTGTATAGCAACGAACCGAACACCAACATCCTGAAAGATTTCTGGGG
CAACTATCTGCTGTACGATAAAGAATATTATCTGCTGAACGTGCTGAAACCGAACAACTTTATTGATC
GCCGTAAAGATAGCACCCTGAGCATTAACAACATTCGTAGCACCATTCTGCTGGCCAACCGTCTGTAT
AGCGGCATTAAAGTGAAAATTCAGCGCGTGAACAATAGCAGCACCAACGATAACCTGGTGCGTAAAAA
CGATCAGGTGTATATCAACTTTGTGGCCAGCAAAACCCACCTGTTTCCGCTGTATGCGGATACCGCGA
CCACCAACAAAGAAAAAACCATTAAAATCAGCAGCAGCGGCAACCGTTTTAACCAGGTGGTGGTGATG
AACAGCGTGGGCAACAACTGTACAATGAACTTCAAAAACAACAACGGCAACAACATTGGCCTGCTGGG
CTTTAAAGCGGATACCGTGGTGGCGAGCACCTGGTATTATACCCACATGCGTGATCATACCAACAGCA
ACGGCTGCTTTTGGAACTTTATTAGCGAAGAACATGGCTGGCAGGAAAAATGA
SEQ ID NO: 9
ATGGCATATCCGTATGATGTTCCGGATTATGCAGTTCGTGGTATTATTACCAGCAAAACCAAAGGTGG
CGGTGGCGGTGGTGGCGGCCCGAAAATCAACAGCTTCAACTATAACGATCCGGTGAACGATCGTACCA
TCCTGTATATTAAACCGGGCGGTTGCCAGGAATTTTACAAAAGCTTCAACATCATGAAAAACATCTGG
ATTATTCCGGAACGTAACGTGATTGGCACCACCCCGCAGGATTTTCATCCGCCGACCAGCCTGAAAAA
CGGCGATAGCAGCTATTATGATCCGAACTATCTGCAGTCTGATGAAGAAAAAGATCGCTTCCTGAAAA
TCGTGACCAAAATCTTCAACCGCATCAACAACAACCTGAGCGGCGGCATTCTGCTGGAAGAACTGAGC
AAAGCGAATCCGTATCTGGGCAACGATAACACTCCAGATAACCAGTTTCATATTGGTGATGCGAGCGC
GGTGGAAATTAAATTTAGCAACGGCTCTCAGGACATTCTGCTGCCGAACGTGATTATTATGGGCGCGG
AACCGGACCTGTTTGAAACCAACAGCAGCAACATTAGCCTGCGTAACAACTATATGCCGAGCAACCAT
GGTTTTGGCAGCATTGCGATTGTGACCTTTAGCCCGGAATATAGCTTTCGCTTCAACGATAACAGCAT
GAACGAATTTATTCAGGACCCGGCGCTGACCCTGATGCACGAGCTGATTCATAGCCTGCATGGCCTGT
ATGGCGCGAAAGGCATTACCACCAAATATACCATCACCCAGAAACAGAATCCGCTGATTACCAACATT
CGTGGCACCAACATTGAAGAATTTCTGACCTTTGGCGGCACCGATCTGAACATTATTACCAGCGCGCA
GAGCAACGATATCTATACCAACCTGCTGGCCGATTATAAAAAAATCGCGTCTAAACTGAGCAAAGTGC
AGGTGAGCAATCCGCTGCTGAATCCGTATAAAGATGTGTTTGAAGCGAAATATGGCCTGGATAAAGAT
GCTAGCGGCATTTATAGCGTGAACATCAACAAATTCAACGACATCTTCAAAAAACTGTATAGCTTTAC
CGAATTTGATCTGGCCACCAAATTTCAGGTGAAATGCCGCCAGACCTATATTGGCCAGTATAAATATT
TTAAACTGAGCAACCTGCTGAACGATAGCATTTACAACATCAGCGAAGGCTATAACATCAACAACCTG
AAAGTGAACTTTCGTGGCCAGAACGCGAATTTAAATCCGCGTATTATTACCCCGATTACCGGCCGTGG
ACTAGTGAAAAAAATTATCCGTTTTTGCGTGCGTGGCATTATCACCAGCAAAACCAAAAGCCTGGTGC
CGCGTGGCAGCAAAGCGTTAAATGATTTATGCATCGAAATCAACAACGGCGAACTGTTTTTTGTGGCG
AGCGAAAACAGCTATAACGATGATAACATCAACACCCCGAAAGAAATTGATGATACCGTGACCAGCAA
TAACAACTACGAAAACGATCTGGATCAGGTGATTCTGAACTTTAACAGCGAAAGCGCACCGGGCCTGT
CTGATGAAAAACTGAACCTGACCATTCAGAACGATGCGTATATCCCGAAATATGATAGCAACGGCACC
AGCGATATTGAACAGCATGATGTGAACGAACTGAACGTGTTTTTTTATCTGGATGCGCAGAAAGTGCC
GGAAGGCGAAAACAACGTGAATCTGACCAGCTCAATTGATACCGCGCTGCTGGAACAGCCGAAAATCT
ATACCTTTTTTAGCAGCGAATTCATCAACAACGTGAACAAACCGGTGCAGGCGGCGCTGTTTGTGAGC
TGGATTCAGCAGGTGCTGGTTGATTTTACCACCGAAGCGAACCAGAAAAGCACCGTGGATAAAATTGC
GGATATTAGCATTGTGGTGCCGTATATTGGCCTGGCCCTGAACATTGGCAACGAAGCGCAGAAAGGCA
ACTTTAAAGATGCGCTGGAACTGCTGGGTGCGGGCATTCTGCTGGAATTTGAACCGGAACTGCTGATT
CCGACCATTCTGGTGTTTACCATCAAAAGCTTTCTGGGCAGCAGCGATAACAAAAACAAAGTGATCAA
AGCGATTAACAACGCGCTGAAAGAACGTGATGAAAAATGGAAAGAAGTGTATAGCTTCATTGTGTCTA
ACTGGATGACCAAAATCAACACCCAGTTCAACAAACGTAAAGAACAAATGTATCAGGCGCTGCAGAAC
CAGGTGAACGCGATTAAAACCATCATCGAAAGCAAATACAACAGCTACACCCTGGAAGAAAAAAACGA
ACTGACCAACAAATATGACATCAAACAAATCGAAAATGAACTGAACCAGAAAGTGAGCATTGCCATGA
ACAACATTGATCGCTTTCTGACCGAAAGCAGCATTAGCTACCTGATGAAACTGATCAACGAAGTGAAA
ATCAACAAACTGCGCGAATATGATGAAAACGTGAAAACCTACCTGCTGAACTATATTATTCAGCATGG
CAGCATTCTGGGCGAAAGCCAGCAAGAACTGAACAGCATGGTTACCGATACCCTGAACAACAGCATTC
CGTTTAAACTGAGCAGCTACACCGATGATAAAATCCTGATCAGCTACTTCAACAAATTCTTCAAACGC
ATCAAAAGCAGCAGCGTGCTGAACATGCGTTATAAAAACGATAAATACGTAGATACCAGCGGCTATGA
TAGCAATATCAACATTAACGGTGATGTGTATAAATACCCGACCAACAAAAACCAGTTCGGCATCTACA
ACGATAAACTGAGCGAAGTGAACATTAGCCAGAACGATTATATCATCTACGATAATAAATATAAAAAC
TTCAGCATCAGCTTTTGGGTGCGTATTCCGAACTACGATAACAAAATCGTGAACGTGAACAACGAATA
CACCATCATTAACTGCATGCGTGATAACAACAGCGGCTGGAAAGTGAGCCTGAACCATAACGAAATCA
TCTGGACCCTGCAGGATAACGCCGGCATTAACCAGAAACTGGCCTTTAACTATGGCAACGCGAACGGC
ATTAGCGATTACATCAACAAATGGATCTTTGTGACCATTACCAACGATCGTCTGGGCGATAGCAAACT
GTATATTAACGGCAACCTGATCGACCAGAAAAGCATTCTGAACCTGGGCAACATTCATGTGAGCGATA
ACATCCTGTTCAAAATTGTGAACTGCAGCTATACCCGTTATATTGGCATCCGCTATTTCAACATCTTC
GATAAAGAACTGGATGAAACCGAAATTCAGACCCTGTATAGCAACGAACCGAACACCAACATCCTGAA
AGATTTCTGGGGCAACTATCTGCTGTACGATAAAGAATATTATCTGCTGAACGTGCTGAAACCGAACA
ACTTTATTGATCGCCGTAAAGATAGCACCCTGAGCATTAACAACATTCGTAGCACCATTCTGCTGGCC
AACCGTCTGTATAGCGGCATTAAAGTGAAAATTCAGCGCGTGAACAATAGCAGCACCAACGATAACCT
GGTGCGTAAAAACGATCAGGTGTATATCAACTTTGTGGCCAGCAAAACCCACCTGTTTCCGCTGTATG
CGGATACCGCGACCACCAACAAAGAAAAAACCATTAAAATCAGCAGCAGCGGCAACCGTTTTAACCAG
GTGGTGGTGATGAACAGCGTGGGCAACAACTGTACAATGAACTTCAAAAACAACAACGGCAACAACAT
TGGCCTGCTGGGCTTTAAAGCGGATACCGTGGTGGCGAGCACCTGGTATTATACCCACATGCGTGATC
ATACCAACAGCAACGGCTGCTTTTGGAACTTTATTAGCGAAGAACATGGCTGGCAGGAAAAATGA

1-28. (canceled)

29. A method for the generation of a recombinant protein with an N-terminal lysine comprising a step of contacting a precursor protein comprising an terminal motif motif X-Lys-linker with an endoprotease which specifically cleaves between X and Lys of the motif, wherein X is an endoprotease recognition sequence, and wherein the linker comprises at least three amino acid residues comprising (i) at least a second Lys residue and/or a Thr residue, and (ii) at least two consecutive Gly residues.

30. The method of claim 29, further comprising a step of obtaining a recombinant nucleic acid encoding the precursor protein by inserting a nucleic acid encoding the N-terminal motif X-Lys-linker into a nucleic acid encoding a parental protein.

31. The method of claim 29, further comprising a step of heterologously expressing a nucleic acid encoding the precursor protein in a host cell before causing or allowing contacting of the precursor protein with the endoprotease.

32. The method of claim 29, wherein the endoprotease is Lys-N from Grifola frondosa.

33. The method of claim 32, wherein the Lys-N is recombinant Lys-N.

34. The method of claim 29, wherein the endoprotease recognition sequence X exhibits the amino acid sequence VRGIITS (SEQ ID NO: 10).

35. The method of claim 29, wherein the linker exhibits an amino acid sequence TKGₙ, wherein n is an integer larger than or equal to 2.

36. The method of claim 29, wherein the linker exhibits an amino acid sequence TKGₙ, wherein n is an integer in a range of from 2 to 12.

37. The method of claim 29, wherein the linker exhibits an amino acid sequence TKGₙ, wherein n is an integer in a range of from 2 to 8.

38. The method of claim 29, wherein the linker exhibits an amino acid sequence TKGₙ, wherein n is an integer selected from the group consisting of 2, 4, and 8.

39. The method of claim 30, wherein the parental protein is a clostridial neurotoxin.

40. The method of claim 39, wherein the clostridial neurotoxin is selected from a Clostridium botulinum neurotoxin of serotype A, B, C, D, E, F, and G, and functional variants thereof.

41. The method of claim 39, wherein the clostridial neurotoxin is selected from Clostridium botulinum neurotoxin serotype A and E, and functional variants thereof.

42. The method of claim 39, wherein the clostridial neurotoxin is Clostridium botulinum neurotoxin serotype E or a functional variant thereof.

43. The method of claim 31, wherein the precursor protein is expressed in E. coli host cells.

44. A precursor protein comprising an N-terminal motif X-Lys-linker, wherein X is an endoprotease recognition sequence, and wherein the linker comprises at least three amino acid residues comprising (i) at least a second Lys residue and/or a Thr residue, and (ii) at least two consecutive Gly residues.

45. The precursor protein of claim 44, wherein the endoprotease recognition sequence X exhibits the amino acid sequence VRGIITS (SEQ ID NO: 10).

46. The precursor protein of claim 44, wherein the linker comprises the amino acid sequence TKGₙ, wherein n is an integer larger than or equal to 2.

47. The precursor protein of claim 44, wherein the linker comprises the amino acid sequence TKGₙ, wherein n is an integer in a range of from 2 to 12.

48. The precursor protein of claim 44, wherein the linker comprises the amino acid sequence TKGₙ, wherein n is an integer in a range of from 2 to 8.

49. The precursor protein of claim 44, wherein the linker comprises the amino acid sequence TKGₙ, wherein n is an integer selected from 2, 4, and 8.

50. The precursor protein of claim 44, which is a clostridial neurotoxin precursor.

51. The precursor protein of claim 50, wherein the clostridial neurotoxin precursor comprises an amino acid sequence as set forth in any one of SEQ ID NOs: 1 to 3.

52. A recombinant protein, wherein the N-terminus of the recombinant protein consists of the sequence Lys-linker, wherein the linker comprises at least three amino acid residues comprising (i) at least a second Lys residue and/or a Thr residue, and (ii) at least two consecutive Gly residues, and wherein the recombinant protein comprises at least 50 amino acid residues, at least 100 amino acid residues, or at least 200 amino acid residues.

53. The recombinant protein of claim 52, wherein the linker comprises the sequence TKGₙ, wherein n is an integer larger than or equal to 2.

54. The recombinant protein of claim 52, wherein the linker comprises the sequence TKGₙ, wherein n is an integer in a range of from 2 to 12.

55. The recombinant protein of claim 52, wherein the linker comprises the sequence TKGₙ, wherein n is an integer in a range of from 2 to 8.

56. The recombinant protein of claim 52, wherein the linker comprises the sequence TKGₙ, wherein n is an integer selected from 2, 4, and 8.

57. The recombinant protein of claim 52, which is a clostridial neurotoxin.

58. The recombinant protein of claim 57, wherein the clostridial neurotoxin comprises an amino acid sequence as set forth in any one of SEQ ID NOs: 4 to 6.

59. A nucleic acid which encodes the precursor protein of claim 44, wherein the nucleic acid comprises a sequence as set forth in any one of SEQ ID NOs: 7 to 9.

60. A method for obtaining the nucleic acid of claim 59, comprising the step of inserting a nucleic acid encoding an N-terminal motif X-Lys-linker into a nucleic acid encoding a parental protein.

61. The method of claim 60, wherein the endoprotease recognition sequence X exhibits the amino acid sequence VRGIITS (SEQ ID NO: 10).

62. The method of claim 60, wherein the linker comprises the amino acid sequence TKGₙ, wherein n is an integer larger than or equal to 2.

63. The method of claim 60, wherein the linker comprises the amino acid sequence TKGₙ, wherein n is an integer in a range of from 2 to 12.

64. The method of claim 60, wherein the linker comprises the amino acid sequence TKGₙ, wherein n is an integer in a range of from 2 to 8.

65. The method of claim 60, wherein the linker comprises the amino acid sequence TKGₙ, wherein n is an integer selected from 2, 4, and 8.

66. The method of claim 60, wherein the parental protein is a clostridial neurotoxin.

67. A vector comprising the nucleic acid of claim 59.

68. A recombinant host cell comprising the nucleic acid of claim 59.

69. A method for generating the precursor protein of claim 44, comprising expressing a nucleic acid encoding the precursor protein in a recombinant host cell and cultivating the recombinant host cell under conditions which result in the expression of the precursor protein.

70. A method for generating the recombinant protein of claim 52, comprising expressing a nucleic acid encoding the recombinant protein in a recombinant host cell and cultivating the recombinant host cell under conditions which result in the expression of the recombinant protein.

71. A pharmaceutical composition comprising the recombinant protein of claim 52.


SEQUENCE LISTING (20 sequences)

SEQ ID NO: 1 - protein, 1284 aa, Artificial Sequence
  clostridial neurotoxin precursor
MAYPYDVPDYAVRGIITSKTKGGPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCVRGIITSKTKSLVPRGSKALNDLCIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 2 - protein, 1286 aa, Artificial Sequence
  clostridial neurotoxin precursor
MAYPYDVPDYAVRGIITSKTKGGGGPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCVRGIITSKTKSLVPRGSKALNDLCIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 3 - protein, 1290 aa, Artificial Sequence
  clostridial neurotoxin precursor
MAYPYDVPDYAVRGIITSKTKGGGGGGGGPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCVRGIITSKTKSLVPRGSKALNDLCIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 4 - protein, 1266 aa, Artificial Sequence
  clostridial neurotoxin with N-terminal lysine
KTKGGPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCVRGIITSKTKSLVPRGSKALNDLCIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 5 - protein, 1268 aa, Artificial Sequence
  clostridial neurotoxin with N-terminal lysine
KTKGGGGPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCVRGIITSKTKSLVPRGSKALNDLCIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 6 - protein, 1272 aa, Artificial Sequence
  clostridial neurotoxin with N-terminal lysine
KTKGGGGGGGGPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCVRGIITSKTKSLVPRGSKALNDLCIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 7 - DNA, 3855 bp, Artificial Sequence
  DNA encoding clostridial neurotoxin precursor
atggcatatccgtatgatgttccggattatgcagttcgtggtattattaccagcaaaaccaaaggtggcccgaaaatcaacagcttcaactataacgatccggtgaacgatcgtaccatcctgtatattaaaccgggcggttgccaggaattttacaaaagcttcaacatcatgaaaaacatctggattattccggaacgtaacgtgattggcaccaccccgcaggattttcatccgccgaccagcctgaaaaacggcgatagcagctattatgatccgaactatctgcagtctgatgaagaaaaagatcgcttcctgaaaatcgtgaccaaaatcttcaaccgcatcaacaacaacctgagcggcggcattctgctggaagaactgagcaaagcgaatccgtatctgggcaacgataacactccagataaccagtttcatattggtgatgcgagcgcggtggaaattaaatttagcaacggctctcaggacattctgctgccgaacgtgattattatgggcgcggaaccggacctgtttgaaaccaacagcagcaacattagcctgcgtaacaactatatgccgagcaaccatggttttggcagcattgcgattgtgacctttagcccggaatatagctttcgcttcaacgataacagcatgaacgaatttattcaggacccggcgctgaccctgatgcacgagctgattcatagcctgcatggcctgtatggcgcgaaaggcattaccaccaaatataccatcacccagaaacagaatccgctgattaccaacattcgtggcaccaacattgaagaatttctgacctttggcggcaccgatctgaacattattaccagcgcgcagagcaacgatatctataccaacctgctggccgattataaaaaaatcgcgtctaaactgagcaaagtgcaggtgagcaatccgctgctgaatccgtataaagatgtgtttgaagcgaaatatggcctggataaagatgctagcggcatttatagcgtgaacatcaacaaattcaacgacatcttcaaaaaactgtatagctttaccgaatttgatctggccaccaaatttcaggtgaaatgccgccagacctatattggccagtataaatattttaaactgagcaacctgctgaacgatagcatttacaacatcagcgaaggctataacatcaacaacctgaaagtgaactttcgtggccagaacgcgaatttaaatccgcgtattattaccccgattaccggccgtggactagtgaaaaaaattatccgtttttgcgtgcgtggcattatcaccagcaaaaccaaaagcctggtgccgcgtggcagcaaagcgttaaatgatttatgcatcgaaatcaacaacggcgaactgttttttgtggcgagcgaaaacagctataacgatgataacatcaacaccccgaaagaaattgatgataccgtgaccagcaataacaactacgaaaacgatctggatcaggtgattctgaactttaacagcgaaagcgcaccgggcctgtctgatgaaaaactgaacctgaccattcagaacgatgcgtatatcccgaaatatgatagcaacggcaccagcgatattgaacagcatgatgtgaacgaactgaacgtgtttttttatctggatgcgcagaaagtgccggaaggcgaaaacaacgtgaatctgaccagctcaattgataccgcgctgctggaacagccgaaaatctataccttttttagcagcgaattcatcaacaacgtgaacaaaccggtgcaggcggcgctgtttgtgagctggattcagcaggtgctggttgattttaccaccgaagcgaaccagaaaagcaccgtggataaaattgcggatattagcattgtggtgccgtatattggcctggccctgaacattggcaacgaagcgcagaaaggcaactttaaagatgcgctggaactgctgggtgcgggcattctgctggaatttgaaccggaactgctgattccgaccattctggtgtttaccatcaaaagctttctgggcagcagcgataacaaaaacaaagtgatcaaagcgattaacaacgcgctgaaagaacgtgatgaaaaatggaaagaagtgtatagcttcattgtgtctaactggatgaccaaaatcaacacccagttcaacaaacgtaaagaacaaatgtatcaggcgctgcagaaccaggtgaacgcgattaaaaccatcatcgaaagcaaatacaacagctacaccctggaagaaaaaaacgaactgaccaacaaatatgacatcaaacaaatcgaaaatgaactgaaccagaaagtgagcattgccatgaacaacattgatcgctttctgaccgaaagcagcattagctacctgatgaaactgatcaacgaagtgaaaatcaacaaactgcgcgaatatgatgaaaacgtgaaaacctacctgctgaactatattattcagcatggcagcattctgggcgaaagccagcaagaactgaacagcatggttaccgataccctgaacaacagcattccgtttaaactgagcagctacaccgatgataaaatcctgatcagctacttcaacaaattcttcaaacgcatcaaaagcagcagcgtgctgaacatgcgttataaaaacgataaatacgtagataccagcggctatgatagcaatatcaacattaacggtgatgtgtataaatacccgaccaacaaaaaccagttcggcatctacaacgataaactgagcgaagtgaacattagccagaacgattatatcatctacgataataaatataaaaacttcagcatcagcttttgggtgcgtattccgaactacgataacaaaatcgtgaacgtgaacaacgaatacaccatcattaactgcatgcgtgataacaacagcggctggaaagtgagcctgaaccataacgaaatcatctggaccctgcaggataacgccggcattaaccagaaactggcctttaactatggcaacgcgaacggcattagcgattacatcaacaaatggatctttgtgaccattaccaacgatcgtctgggcgatagcaaactgtatattaacggcaacctgatcgaccagaaaagcattctgaacctgggcaacattcatgtgagcgataacatcctgttcaaaattgtgaactgcagctatacccgttatattggcatccgctatttcaacatcttcgataaagaactggatgaaaccgaaattcagaccctgtatagcaacgaaccgaacaccaacatcctgaaagatttctggggcaactatctgctgtacgataaagaatattatctgctgaacgtgctgaaaccgaacaactttattgatcgccgtaaagatagcaccctgagcattaacaacattcgtagcaccattctgctggccaaccgtctgtatagcggcattaaagtgaaaattcagcgcgtgaacaatagcagcaccaacgataacctggtgcgtaaaaacgatcaggtgtatatcaactttgtggccagcaaaacccacctgtttccgctgtatgcggataccgcgaccaccaacaaagaaaaaaccattaaaatcagcagcagcggcaaccgttttaaccaggtggtggtgatgaacagcgtgggcaacaactgtacaatgaacttcaaaaacaacaacggcaacaacattggcctgctgggctttaaagcggataccgtggtggcgagcacctggtattatacccacatgcgtgatcataccaacagcaacggctgcttttggaactttattagcgaagaacatggctggcaggaaaaatga

SEQ ID NO: 8 - DNA, 3861 bp, Artificial Sequence
  DNA encoding clostridial neurotoxin precursor 2
atggcatatccgtatgatgttccggattatgcagttcgtggtattattaccagcaaaaccaaaggtggtggcggcccgaaaatcaacagcttcaactataacgatccggtgaacgatcgtaccatcctgtatattaaaccgggcggttgccaggaattttacaaaagcttcaacatcatgaaaaacatctggattattccggaacgtaacgtgattggcaccaccccgcaggattttcatccgccgaccagcctgaaaaacggcgatagcagctattatgatccgaactatctgcagtctgatgaagaaaaagatcgcttcctgaaaatcgtgaccaaaatcttcaaccgcatcaacaacaacctgagcggcggcattctgctggaagaactgagcaaagcgaatccgtatctgggcaacgataacactccagataaccagtttcatattggtgatgcgagcgcggtggaaattaaatttagcaacggctctcaggacattctgctgccgaacgtgattattatgggcgcggaaccggacctgtttgaaaccaacagcagcaacattagcctgcgtaacaactatatgccgagcaaccatggttttggcagcattgcgattgtgacctttagcccggaatatagctttcgcttcaacgataacagcatgaacgaatttattcaggacccggcgctgaccctgatgcacgagctgattcatagcctgcatggcctgtatggcgcgaaaggcattaccaccaaatataccatcacccagaaacagaatccgctgattaccaacattcgtggcaccaacattgaagaatttctgacctttggcggcaccgatctgaacattattaccagcgcgcagagcaacgatatctataccaacctgctggccgattataaaaaaatcgcgtctaaactgagcaaagtgcaggtgagcaatccgctgctgaatccgtataaagatgtgtttgaagcgaaatatggcctggataaagatgctagcggcatttatagcgtgaacatcaacaaattcaacgacatcttcaaaaaactgtatagctttaccgaatttgatctggccaccaaatttcaggtgaaatgccgccagacctatattggccagtataaatattttaaactgagcaacctgctgaacgatagcatttacaacatcagcgaaggctataacatcaacaacctgaaagtgaactttcgtggccagaacgcgaatttaaatccgcgtattattaccccgattaccggccgtggactagtgaaaaaaattatccgtttttgcgtgcgtggcattatcaccagcaaaaccaaaagcctggtgccgcgtggcagcaaagcgttaaatgatttatgcatcgaaatcaacaacggcgaactgttttttgtggcgagcgaaaacagctataacgatgataacatcaacaccccgaaagaaattgatgataccgtgaccagcaataacaactacgaaaacgatctggatcaggtgattctgaactttaacagcgaaagcgcaccgggcctgtctgatgaaaaactgaacctgaccattcagaacgatgcgtatatcccgaaatatgatagcaacggcaccagcgatattgaacagcatgatgtgaacgaactgaacgtgtttttttatctggatgcgcagaaagtgccggaaggcgaaaacaacgtgaatctgaccagctcaattgataccgcgctgctggaacagccgaaaatctataccttttttagcagcgaattcatcaacaacgtgaacaaaccggtgcaggcggcgctgtttgtgagctggattcagcaggtgctggttgattttaccaccgaagcgaaccagaaaagcaccgtggataaaattgcggatattagcattgtggtgccgtatattggcctggccctgaacattggcaacgaagcgcagaaaggcaactttaaagatgcgctggaactgctgggtgcgggcattctgctggaatttgaaccggaactgctgattccgaccattctggtgtttaccatcaaaagctttctgggcagcagcgataacaaaaacaaagtgatcaaagcgattaacaacgcgctgaaagaacgtgatgaaaaatggaaagaagtgtatagcttcattgtgtctaactggatgaccaaaatcaacacccagttcaacaaacgtaaagaacaaatgtatcaggcgctgcagaaccaggtgaacgcgattaaaaccatcatcgaaagcaaatacaacagctacaccctggaagaaaaaaacgaactgaccaacaaatatgacatcaaacaaatcgaaaatgaactgaaccagaaagtgagcattgccatgaacaacattgatcgctttctgaccgaaagcagcattagctacctgatgaaactgatcaacgaagtgaaaatcaacaaactgcgcgaatatgatgaaaacgtgaaaacctacctgctgaactatattattcagcatggcagcattctgggcgaaagccagcaagaactgaacagcatggttaccgataccctgaacaacagcattccgtttaaactgagcagctacaccgatgataaaatcctgatcagctacttcaacaaattcttcaaacgcatcaaaagcagcagcgtgctgaacatgcgttataaaaacgataaatacgtagataccagcggctatgatagcaatatcaacattaacggtgatgtgtataaatacccgaccaacaaaaaccagttcggcatctacaacgataaactgagcgaagtgaacattagccagaacgattatatcatctacgataataaatataaaaacttcagcatcagcttttgggtgcgtattccgaactacgataacaaaatcgtgaacgtgaacaacgaatacaccatcattaactgcatgcgtgataacaacagcggctggaaagtgagcctgaaccataacgaaatcatctggaccctgcaggataacgccggcattaaccagaaactggcctttaactatggcaacgcgaacggcattagcgattacatcaacaaatggatctttgtgaccattaccaacgatcgtctgggcgatagcaaactgtatattaacggcaacctgatcgaccagaaaagcattctgaacctgggcaacattcatgtgagcgataacatcctgttcaaaattgtgaactgcagctatacccgttatattggcatccgctatttcaacatcttcgataaagaactggatgaaaccgaaattcagaccctgtatagcaacgaaccgaacaccaacatcctgaaagatttctggggcaactatctgctgtacgataaagaatattatctgctgaacgtgctgaaaccgaacaactttattgatcgccgtaaagatagcaccctgagcattaacaacattcgtagcaccattctgctggccaaccgtctgtatagcggcattaaagtgaaaattcagcgcgtgaacaatagcagcaccaacgataacctggtgcgtaaaaacgatcaggtgtatatcaactttgtggccagcaaaacccacctgtttccgctgtatgcggataccgcgaccaccaacaaagaaaaaaccattaaaatcagcagcagcggcaaccgttttaaccaggtggtggtgatgaacagcgtgggcaacaactgtacaatgaacttcaaaaacaacaacggcaacaacattggcctgctgggctttaaagcggataccgtggtggcgagcacctggtattatacccacatgcgtgatcataccaacagcaacggctgcttttggaactttattagcgaagaacatggctggcaggaaaaatga

SEQ ID NO: 9 - DNA, 3873 bp, Artificial Sequence
  DNA encoding clostridial neurotoxin precursor 3
atggcatatccgtatgatgttccggattatgcagttcgtggtattattaccagcaaaaccaaaggtggcggtggcggtggtggcggcccgaaaatcaacagcttcaactataacgatccggtgaacgatcgtaccatcctgtatattaaaccgggcggttgccaggaattttacaaaagcttcaacatcatgaaaaacatctggattattccggaacgtaacgtgattggcaccaccccgcaggattttcatccgccgaccagcctgaaaaacggcgatagcagctattatgatccgaactatctgcagtctgatgaagaaaaagatcgcttcctgaaaatcgtgaccaaaatcttcaaccgcatcaacaacaacctgagcggcggcattctgctggaagaactgagcaaagcgaatccgtatctgggcaacgataacactccagataaccagtttcatattggtgatgcgagcgcggtggaaattaaatttagcaacggctctcaggacattctgctgccgaacgtgattattatgggcgcggaaccggacctgtttgaaaccaacagcagcaacattagcctgcgtaacaactatatgccgagcaaccatggttttggcagcattgcgattgtgacctttagcccggaatatagctttcgcttcaacgataacagcatgaacgaatttattcaggacccggcgctgaccctgatgcacgagctgattcatagcctgcatggcctgtatggcgcgaaaggcattaccaccaaatataccatcacccagaaacagaatccgctgattaccaacattcgtggcaccaacattgaagaatttctgacctttggcggcaccgatctgaacattattaccagcgcgcagagcaacgatatctataccaacctgctggccgattataaaaaaatcgcgtctaaactgagcaaagtgcaggtgagcaatccgctgctgaatccgtataaagatgtgtttgaagcgaaatatggcctggataaagatgctagcggcatttatagcgtgaacatcaacaaattcaacgacatcttcaaaaaactgtatagctttaccgaatttgatctggccaccaaatttcaggtgaaatgccgccagacctatattggccagtataaatattttaaactgagcaacctgctgaacgatagcatttacaacatcagcgaaggctataacatcaacaacctgaaagtgaactttcgtggccagaacgcgaatttaaatccgcgtattattaccccgattaccggccgtggactagtgaaaaaaattatccgtttttgcgtgcgtggcattatcaccagcaaaaccaaaagcctggtgccgcgtggcagcaaagcgttaaatgatttatgcatcgaaatcaacaacggcgaactgttttttgtggcgagcgaaaacagctataacgatgataacatcaacaccccgaaagaaattgatgataccgtgaccagcaataacaactacgaaaacgatctggatcaggtgattctgaactttaacagcgaaagcgcaccgggcctgtctgatgaaaaactgaacctgaccattcagaacgatgcgtatatcccgaaatatgatagcaacggcaccagcgatattgaacagcatgatgtgaacgaactgaacgtgtttttttatctggatgcgcagaaagtgccggaaggcgaaaacaacgtgaatctgaccagctcaattgataccgcgctgctggaacagccgaaaatctataccttttttagcagcgaattcatcaacaacgtgaacaaaccggtgcaggcggcgctgtttgtgagctggattcagcaggtgctggttgattttaccaccgaagcgaaccagaaaagcaccgtggataaaattgcggatattagcattgtggtgccgtatattggcctggccctgaacattggcaacgaagcgcagaaaggcaactttaaagatgcgctggaactgctgggtgcgggcattctgctggaatttgaaccggaactgctgattccgaccattctggtgtttaccatcaaaagctttctgggcagcagcgataacaaaaacaaagtgatcaaagcgattaacaacgcgctgaaagaacgtgatgaaaaatggaaagaagtgtatagcttcattgtgtctaactggatgaccaaaatcaacacccagttcaacaaacgtaaagaacaaatgtatcaggcgctgcagaaccaggtgaacgcgattaaaaccatcatcgaaagcaaatacaacagctacaccctggaagaaaaaaacgaactgaccaacaaatatgacatcaaacaaatcgaaaatgaactgaaccagaaagtgagcattgccatgaacaacattgatcgctttctgaccgaaagcagcattagctacctgatgaaactgatcaacgaagtgaaaatcaacaaactgcgcgaatatgatgaaaacgtgaaaacctacctgctgaactatattattcagcatggcagcattctgggcgaaagccagcaagaactgaacagcatggttaccgataccctgaacaacagcattccgtttaaactgagcagctacaccgatgataaaatcctgatcagctacttcaacaaattcttcaaacgcatcaaaagcagcagcgtgctgaacatgcgttataaaaacgataaatacgtagataccagcggctatgatagcaatatcaacattaacggtgatgtgtataaatacccgaccaacaaaaaccagttcggcatctacaacgataaactgagcgaagtgaacattagccagaacgattatatcatctacgataataaatataaaaacttcagcatcagcttttgggtgcgtattccgaactacgataacaaaatcgtgaacgtgaacaacgaatacaccatcattaactgcatgcgtgataacaacagcggctggaaagtgagcctgaaccataacgaaatcatctggaccctgcaggataacgccggcattaaccagaaactggcctttaactatggcaacgcgaacggcattagcgattacatcaacaaatggatctttgtgaccattaccaacgatcgtctgggcgatagcaaactgtatattaacggcaacctgatcgaccagaaaagcattctgaacctgggcaacattcatgtgagcgataacatcctgttcaaaattgtgaactgcagctatacccgttatattggcatccgctatttcaacatcttcgataaagaactggatgaaaccgaaattcagaccctgtatagcaacgaaccgaacaccaacatcctgaaagatttctggggcaactatctgctgtacgataaagaatattatctgctgaacgtgctgaaaccgaacaactttattgatcgccgtaaagatagcaccctgagcattaacaacattcgtagcaccattctgctggccaaccgtctgtatagcggcattaaagtgaaaattcagcgcgtgaacaatagcagcaccaacgataacctggtgcgtaaaaacgatcaggtgtatatcaactttgtggccagcaaaacccacctgtttccgctgtatgcggataccgcgaccaccaacaaagaaaaaaccattaaaatcagcagcagcggcaaccgttttaaccaggtggtggtgatgaacagcgtgggcaacaactgtacaatgaacttcaaaaacaacaacggcaacaacattggcctgctgggctttaaagcggataccgtggtggcgagcacctggtattatacccacatgcgtgatcataccaacagcaacggctgcttttggaactttattagcgaagaacatggctggcaggaaaaatga

SEQ ID NO: 10 - protein, 7 aa, Grifola frondosa
VRGIITS

SEQ ID NO: 11 - protein, 7 aa, Artificial sequence
  artificial cleavage sequence for Lys-N
MKGGINS

SEQ ID NO: 12 - protein, 21 aa, Artificial Sequence
  artificial cleavage sequence for Lys-N
MAYPYDVPDYAKGGGGPKINS

SEQ ID NO: 13 - protein, 26 aa, Artificial Sequence
  artificial cleavage sequence for Lys-N
MAYPYDVPDYAKGGGGKGGGGPKINS

SEQ ID NO: 14 - protein, 30 aa, Artificial Sequence
  artificial cleavage sequence for Lys-N
MAYPYDVPDYAVRGIITSKTKGGGGPKINS

SEQ ID NO: 15 - protein, 34 aa, Artificial Sequence
  artificial cleavage sequence for Lys-N
MAYPYDVPDYAVRGIITSKTKGGGGGGGGPKINS

SEQ ID NO: 16 - protein, 28 aa, Artificial Sequence
  artificial cleavage sequence for Lys-N
MAYPYDVPDYAVRGIITSKGGGGPKINS

SEQ ID NO: 17 - protein, 24 aa, Artificial Sequence
  artificial cleavage sequence for Lys-N
MAYPYDVPDYAVRGIITSKPKINS

SEQ ID NO: 18 - protein, 25 aa, Artificial Sequence
  artificial cleavage sequence for Lys-N
MAYPYDVPDYAVRGIITSKTPKINS

SEQ ID NO: 19 - protein, 26 aa, Artificial Sequence
  artificial cleavage sequence for Lys-N
MAYPYDVPDYAVRGIITSKTKPKINS

SEQ ID NO: 20 - protein, 28 aa, Artificial Sequence
  artificial cleavage sequence for Lys-N
MAYPYDVPDYAVRGIITSKTKGGPKINS